(19) United States
(12) Patent Application Publication
(10) Pub. No.: US 2014/0343259 A1
(43) Pub. Date: Nov. 20, 2014

(54) PROTEIN PRODUCT

(71) Applicants: James R. Bleyer, Maumee, OH (US); Jennifer Aurandt, Brighton, MI (US); Thomas J. Czartoski, Tecumseh, MI (US)

(72) Inventors: James R. Bleyer, Maumee, OH (US); Jennifer Aurandt, Brighton, MI (US); Thomas J. Czartoski, Tecumseh, MI (US)

(73) Assignee: VALICOR, INC., Dexter, MI (US)

(21) Appl. No.: 14/449,473

(22) Filed: Aug. 1, 2014

Related U.S. Application Data
(63) Continuation-in-part of application No. 13/922,497, filed on Jun. 20, 2013.
(60) Provisional application No. 61/662,019, filed on Jun. 20, 2012.

(51) Int. Cl. C07K 1/36; A23J 1/00; C07K 1/34

(52) U.S. Cl. CPC C07K 1/36; C07K 1/34; A23J 1/006
USPC 530/414; 530/350; 530/412

(57) ABSTRACT

A protein product recovered from a fermentation process including a protein content of 45.0% or more calculated by weight of dry matter, a glycerol content of 1.0% or less calculated by weight of dry matter, and a mineral nutrient content of 6.0% or less calculated by weight of dry matter. A method for recovering a protein product by heating fermentation stillage to 200 degrees F.-350 degrees F., altering the physicochemical properties of the stillage to enable facile separation of the stillage, and separating a phase enriched in protein. Protein products, protein paste, high protein meal, and proteinaceous agglomerates recovered and formed.

### BACKGROUND OF THE INVENTION

1. Technical Field

The present invention generally relates to a protein product and methods of producing such product. More particularly, the present invention relates to a protein product recovered from a fermentation process as well as methods of recovering such product.

2. Background Art

Demand for suitable sources of animal feed has risen in recent years. One of the major driving forces behind the increase in this demand is the increase in the worldwide population of the middle class. According to the Wolfensohn Center for Development at Brookings, the Asia Pacific region's middle class will increase by 614% from 2009 to 2030. The Mediterranean and North Africa region's middle class will increase by 222% during the same period. As populations move from low economic class to middle economic class, their diet transitions from one of primarily grain protein to one of animal protein. This increased consumption of animal proteins has strained current protein supplies for animal feeds.

Animal feed must satisfy the nutritional needs of the species being fed. Animal feed must provide essential amino acids: histidine, isoleucine, leucine, lysine, methionine, phenylalanine, threonine, tryptophan, and valine. It must supply the required metabolizable energy, along with essential vitamins and minerals. Additional components are also desired for certain species. For example, laying hens are often fed lutein so that they produce an egg that has a more intense yellow yolk. Nutritionists place a higher value on feed material that contains a combination of these essential components.

Potential sources for an animal feed product are the byproducts of fermentation. Fermentation is the biological process by which microorganism fermentation agents convert carbohydrates to alcohols, acids and/or other products. Bacteria and yeast are the predominant microorganism fermentation agents used for such fermentations; however, algae, fungi and other micro-organisms can also be used. The most common fermentation processes utilize sugars as the primary carbohydrate source.

The sugars can be simple sugars from sugar producing plants such as sugar cane, sugar beets and sweet sorghum. In the United States, most of the sugar used in fermentation is derived from grain starch. For example, ethanol is produced in a fermentation process by hydrolyzing starch to glucose and then converting the glucose to alcohol. Historically, corn is the predominant grain used to produce ethanol in the United States, but other grains, such as milo and wheat have also been used.

Dry grinding is predominantly used to produce ethanol from corn and other grains through fermentation (shown generally in FIG. 1, labeled “Prior Art”). The grain is milled to flour (10), slurried, and treated with enzymes (12) to convert the starch to sugars.

The yeast Saccharomyces cerevisiae (S. cerevisiae) is added to convert the majority of the sugars to ethanol (14), but it also produces other metabolites such as glycerol and organic acids. These metabolites are not the targeted products of the fermentation process and are known as “off product metabolites.” Zhang and Chen estimate that 5% of the available sugars in an ethanol fermentation process are converted to glycerol. Some of the sugars in the fermentation process are used by the fermentation agent to grow and multiply. Off product metabolites and fermentation agent biomass have implications on the suitability of fermentation byproducts as a protein product.

The fermented mash is distilled (16) to separate and recover ethanol. The residual aqueous slurry of spent grains, referred to as whole stillage (18), contains corn germ, corn bran, corn oil, unconverted starch, unfermented sugars, cells of the fermentation agent, off product metabolites, proteins of corn and the fermentation agent and other suspended and dissolved solids. The whole stillage stream is generally separated (20) into wet distiller's grain (WDG) (22) and thin stillage (24). This separation can be through centrifugation, decantation, filtration or any other suitable means. The wet distiller's grains, also known as wet cake, can be dried to produce Dry Distillers Grain (DDG). A portion of the thin stillage, referred to as backset (26), is recycled back to the front end of the ethanol process as make up water. The remaining thin stillage (28) is fed to an evaporator (30), evaporated to a syrup (32) containing concentrated solubles and fine suspended solids, added to the wet distiller's grains and dried (34) as Dried Distillers Grains with Solubles (DDGS) (36). WDG, DDG, and DDGS are important co-products that are critical to the economic viability of the ethanol process. For convenience, DDG, WDG, and DDGS are referred to as distiller's grain products. Typical commercial DDGS from a dry-grind corn ethanol plant contain about 28-30% crude protein by weight of dry matter.

Distiller's grain products have many positive attributes as an animal feed product. Because the starch contained in the grain is converted to the fermentation product, the protein of the grain is concentrated. Distiller's grain products also contain other nutritional components desirable in animal feed such as lipids, minerals, and vitamins. The lipids contain soluble compounds such as lutein and zeaxanthin. The minerals sulfur, phosphorus, potassium, magnesium, calcium, sodium, iron, manganese, copper, and zinc, collectively known as nutritional minerals, are useful in an animal feed component, within limits.

Distiller's grain products also contain the yeast biomass produced during fermentation. Yeast biomass is desirable in an animal feed product because of their protein, beta-glucan, mannose, and vitamin content. Brewers yeast can contain up to 60% protein by weight and are enriched in the essential amino acid lysine. The yeast cell wall consists of the oligosaccharides beta-glucan and mannose. Beta-glucans are immune-stimulatory due to their nature of aiding in antigen presentation. In addition, mannose moieties function as a growth medium for the bacteria flora found in the gut. The vitamins biotin, niacin, pantothenic acid, and thiamine are enriched in yeast cells that aid in macromolecule synthesis. Yeast biomass has a high nucleic acid content, which can be beneficial for animal immune support and development; however, it also results in nucleic acid metabolites in monogastric animals. One metabolite in particular, uric acid, if produced in excess can cause blood acidification causing debility, cloacitis and cannibalism. Therefore, the yeast component of an animal feed should fall within specific concentrations.

Distiller's grain products produced with current art have limitations. While the protein in the distiller's grain products is concentrated as compared to the source grain, the product is considered a medium protein animal feed, with protein levels typically less than 30%. In addition, distiller's grain products are deficient in certain amino acids and can require the addition of expensive synthetic amino acids. The fiber present in the material is non-digestible by mono-gastric animals and provides no nutrition to them.

Distiller's grain products also contain high levels of off product metabolites, specifically glycerol and organic acids that detract from their value as an animal feed. These metabolites attract moisture and affect the shelf life and material handling characteristics of the product.

The Equilibrium Moisture Content (EMC), or measure of the amount of moisture that can be absorbed by the material, is calculated based upon the chemical composition. EMC is calculated based on water chemical potential between the component and the surrounding air described by Kingley, et al. for DDGS as:

Xₜ=Xᵥ+W_{f}X_{f}+WₛXₛ+WₚXₚ+W_{g}X_{g}

wherein X is the equilibrium moisture content, dry basis (decimal); W is the weight fraction of component, dry basis (decimal); t is the total (combined effect of all components); v is the vacuole (sugars and minerals); f is the fiber (cellulose); s is the starch; p is the protein; g is the glycerol, dry basis (decimal). Based upon above equations a decrease in the amount of glycerol and ions, such as nutrient minerals, decreases the EMC.

High levels of moisture cause caking, arching and other problems in material handling. A decrease in the EMC decreases caking and arching. A decrease in the levels of glycerol and nutrient minerals decreases the EMC thereby improving material handling.

Moisture also affects the shelf-life of the material. Free water provides a water source for microorganisms such as fungi and bacteria. The more free water associated with the feed material the greater the tendency for spoilage. A decrease in glycerol and nutrient minerals decreases the EMC thereby extending the shelf life of the material. Producers of distiller's grain products will often over dry their product in an effort to compensate for increased levels of nutrient minerals, glycerol and other off product metabolites, increasing energy consumption and decreasing product throughput.

Various processes for recovering a higher protein product from a fermentation process are currently known in the art. These processes use various methods of physical separation. The resultant slurry from grain fermentation is a complex mixture of soluble and insoluble components that are inherently difficult to separate and dewater. Processes that attempt to do so can be expensive, inefficient or even dangerous. Effective separation of protein requires conditioning of the slurry to induce physicochemical changes that alter the hydrophilic nature of the protein exposing the hydrophobic core resulting in agglomeration. The particles then reject the water, water-soluble components and other components of the liquid phase. The recovered protein product is of higher purity and superior quality. In the processes of the prior art, the protein is not transformed but merely separated. The effect of off-product metabolites and minerals are not recognized and their deleterious effect on the suitability of the product is not mitigated.

Disclosed in the prior art are methods that operate on the front-end, so-called “front-end fractionation” processes that increase protein concentration of the distiller's grain product. POET Research Inc. in application EP 2281898 A1 discloses a front-end dry fractionation method followed by raw starch hydrolysis (i.e. without liquefaction) and a high protein distiller's grain containing at least 40% protein and an animal feed derived thereof. However, the effects of the off-product metabolites and minerals are not contemplated. In fact, in one embodiment, the condensed distiller's solubles, which contain high levels of off-product metabolites and minerals, are added to the high protein distiller's grain.

Anderson, et al. in U.S. Pat. No. 7,820,418 assigned to GrainValue LLC disclose a process in which corn is fractionated to achieve separate bran, germ, and a starch-and-protein containing fraction. The bran fraction is hydrolyzed to produce a bran hydrolysate containing cellulosic sugars. The starch-and-protein containing fraction is treated separately to hydrolyze the starch and the insoluble protein is separated from the starch hydrolysate. After anaerobic fermentation of the starch hydrolysate, the anaerobic fermentate is separated into ethanol, a yeast-containing stream and a mixture of water and anaerobic fermentation by-products. The unfermented by-products and yeast streams and a portion of the bran hydrolysate stream may be used to grow additional yeast aerobically. The aerobic fermentate can be separated into a second stream of aerobically fermented yeast and water. Thus protein isolated from the starch hydrolysate and the protein contained in aerobically grown yeast, are potential high protein streams produced in this process. Aerobic fermentation of bran hydrolysate is expensive due to high installed capital for pre-treatment of bran cellulose and aerated fermentation. Operating costs are significant due to cellulose hydrolyzing enzymes and chemicals if needed for pre-treatment of the cellulose and hemi-cellulose components of bran. Glycerol and organic acid content will be marginally reduced in aerobic fermentation. Furthermore, it does not degrade or reduce the presence of minerals and non-organic soluble solids.

Several back-end fractionation processes that result in high protein DDG or DDGS have been proposed. Y. V. Wu, et al. (Cereal Chemistry 58(4) 343-347, 1981) and Y. V. Wu (Cereal Chemistry, 66(6, 506-509, 1989) describe the separation and isolation of a high protein fraction from corn ethanol whole stillage by a sequential process of filtration, centrifugal separation and dewatering and then drying, thereby achieving dry basis protein concentrations in the range of 42-57% depending on corn variety. Wu, et al. (1981) and Wu (1989) do not contemplate the effects of the off-product metabolites and minerals.

V. Erlich in U.S. Pat. No. 2,466,913 discloses a process for treating and recovering salts and minerals from clarified thin stillage via ion exchange adsorption methods. During the clarification process, whole stillage is separated by filtration and the resulting thin stillage is further clarified by centrifugation to give a protein containing fraction and clarified thin stillage. However, the removal of salts and minerals occurs after isolation of the protein fraction and does not affect the protein-containing fraction. Hence no mitigation of inorganic solubles is afforded by the method of Erlich.

K. Schwenke et al. in W02005/029974 A1 assigned to Heineken Technical Services B.V. discloses the separation of a proteinaceous juice from fermentation residue (stillage) by a process including sieving to separate fibers from protein-containing filtrate, followed by weight separation methods, e.g. settling and centrifugation to isolate a protein fraction from the sieve filtrate. The protein concentrate can be dried and compositions having 40 -80 wt % protein on a dry basis are disclosed. Components other than protein, fat, fiber and amino acids are not considered in this art and the effects of the off-product metabolites and minerals are not contemplated.

P. E. V. Williams in U.S. Patent Application Publication No. 2012/0121565 A1 assigned to AB Agri Ltd. discloses the recovery of a fermentation agent, i.e. a yeast paste, from whole stillage by a two-step separation process. In the first step, whole stillage is separated into a first stream containing unfermented organic material and a second stream of protein-containing fermentation agent (i.e. yeast). The second stream is subjected to a second separation step to recover a third stream containing the fermentation agent and a fourth stream of aqueous solution. Various means of mechanical separation are disclosed including a decanting centrifuge for the first separation and a disk stack centrifuge for the second separation. The protein stream can be further dewatered by a dewatering device such as a filter press. It is disclosed that the protein-containing fermentation agent in animal feed compositions has utility. However, the effects of the off-product metabolites and minerals are not contemplated.

In another example of post-fermentation physical separation, C. Y. Lee in U.S. Patent Application Publication No. 2012/0064213 A1 assigned to Fluid Quip Inc. discloses a method for producing a high protein corn meal from a whole stillage byproduct comprising separating a whole stillage byproduct into an insoluble solids portion and a thin stillage portion via constituent particle size; separating the thin stillage portion into a protein portion and a water soluble solids portion via constituent weights; dewatering the protein portion; and drying the dewatered protein portion to define a high protein corn meal that includes at least 40 wt % protein on a dry basis. However, other components of the high protein meal are not considered in this art and the effects of the off-product metabolites and minerals are not contemplated.

Kohl, et al. in U.S. Patent Application Publication No. 2013/0165678 A1 disclose a method of clarifying a thin stillage product in a mechanical processor to produce a fine suspended solids stream and clarified thin stillage. By example Kohl, et al. show that the fine suspended solids stream derived from thin stillage has about 40 wt % protein on a dry matter basis and may be dried to produce dry distiller's solubles containing single cell proteins. Components other than protein and suspended solids are not considered in this art and the effects of the off-product metabolites and minerals are not contemplated.

J. A. Bootsma in European patent application EP2699655(A1) or international application WO2012145230A1 assigned to POET Research Inc., disclose a system for fractionating whole stillage comprising a first separator configured to separate whole stillage to thin stillage and a high fiber wet cake; a three phase separator configured to separate the thin stillage to a clarified stillage, an oil emulsion and a protein paste; a first dryer to dry the high fiber wet cake to generate a high fiber animal feed product; a second dryer to dry the protein paste to generate a high protein animal feed product containing at least 45% by weight protein on a dry basis. Further disclosed is the whole stillage being separated using a screening centrifuge, and the thin stillage liquid portion being separated using a three phase disk nozzle centrifuge. Components of the high protein animal product other than protein, fat and fiber are not considered in this art and the effects of off-product metabolites are not contemplated.

R. Fritz and L. Sander in U.S. Pat. No. 7,829,680 assigned to ProGold Plus Inc. disclose a method for protein isolation as a co-product of alcohol production, essentially comprising: directing a grain product such as whole stillage, over a plurality of screens, collecting a fiber-containing portion and allowing a protein-containing portion and an oil-containing portion of the grain product to pass through the screens; directing the protein-containing portion and the oil-containing portion over a finishing screen; centrifuging the protein-containing portion and the oil-containing portion to isolate a protein fraction, an oil fraction, and a water and minerals fraction; and recovering the isolated protein fraction. The application further claims drying the isolated protein fraction. Components other than protein are not considered in this art and the effects of off-product metabolites are not contemplated.

Chemically assisted means of collecting suspended solids from the thin stillage of a dry grind ethanol facility are disclosed by Schiemann, et al. in U.S. Pat. No. 7,497,955 assigned to Nalco Corp. The process of Schiemann, et al. includes the addition to thin stillage of certain anionic polymeric flocculating agents, followed by separation, preferably with a low-shear device such as a settling tank. The process can further include the addition of cationic polymeric coagulants and “micro-particulate settling aids” (e.g. clays, micro-sands, colloidal silica) to aid in the coagulation and solids removal process. The disclosed process does not teach the composition of the wet settled solids stream or anticipate further isolation of a dry high protein stream thereof, but rather suggests conventional disposition such as concentration and evaporation to syrup or sending to the plant's feed dryer. This process is also disadvantaged by the requirement for costly chemical additives and concerns regarding suitability of the additives in animal feeds.

Other inventors have disclosed back-end processes that recover the protein from the beer prior to distillation. S. Redford in international application WO2014014683 A1 assigned to POET Research Inc. discloses feed compositions and methods of making feed compositions from a beer composition of starch-based ethanol process. The feed compositions may include, on a dry weight basis, crude protein in an amount of about 40 wt % or greater, crude fat in an amount of up to about 20 wt %, neutral detergent fiber in an amount of up to about 20 wt %, and lysine in an amount of about 2.55 wt % or greater. The methods involve separating a beer composition, prior to distillation, into bulk solids and a fine solids, and removing liquid from the fine solids, in which the separating and removing are performed in a manner that reduces the total heat exposure of the fine solids as compared to processes relying on distillation to separate beer components and/or cooking to saccharify starch.

F. T. Barrows, et al. in U.S. Pat. No. 8,382,677, now assigned to Montana Microbial LLC, describes a process to produce a protein concentrate from a starch containing grain or oil seed by separation of a fermentation slurry, prior to distillation, into a liquid fraction and a protein containing solids fraction by any suitable separation method and air drying the solids fraction at a temperature that does not denature or damage proteins, said temperature disclosed as typically 40-100 degrees C. Again, the pre-distillation methods of Redford (WO2014014683) and Barrows, et al. (U.S. Pat. No. 8,382,677) do not contemplate the impact of solubles such as glycerol and salts nor provides any means to mitigate solubles.

A number of back-end methods have been developed involving heat-treating stillage for the purposes of recovery of fermentation by-products, such as distiller's grains and syrup and especially oil. C. Brown in U.S. Pat. Nos. 2,216,904 and 2,216,905, assigned to The Sharples Corp., discloses the pre-treatment of “fermentation slop” by pressurized heat treatment to enhance agglomeration of slop solids, subsequently separating solids by filtration, settling or centrifugation and thereafter recovering oil from said solids. In related U.S. Pat. No. 2,263,608, Brown further discloses coarse 250 micron filtration or screening of “fermentation slop” followed by heating the filtrate to elevated temperatures and pressures, thus converting the slop to improve settling of solids and downstream filtration, allowing solids to settle, decanting a clear liquid and subsequently treating the settled solids with an alkaline reagent to precipitate additional solids, filtering the precipitated solids and re-acidifying the filtrate with the acidic clear liquid from settling, and evaporating the re-acidified filtrate to produce a syrup. Brown '608 further discloses precipitation of protein material from thin slop by heat treating and combining thin slop containing protein precipitate with size-reduced (and optionally heat treated) thick slop solids, said size-reduced solids serving as a filter aid in a subsequent filtration step. Collected solids in any of the embodiments of the Brown '608 patent can be used as animal feed. However, a high protein-containing fraction is not isolated by the art described in Brown '904 or '608 and the protein content of the animal feed cannot be higher than the protein content of the thick slop on a dry solids basis.

U.S. Patent Application Publication No. 2009/0250412 and U.S. Pat. No. 7,608,729 to Winsness, et al., disclose methods for recovering oil from stillage concentrate including oil resulting from a process used for producing ethanol from corn. Winsness, et al. generally believe that filtration increases operating costs and therefore focus on separation by heating. In one embodiment, the method includes heating the stillage concentrate to a temperature sufficient to at least partially separate, or unbind the oil. The heating step includes heating to a temperature above 212 degrees F. but less than about 250 degrees F. The method also includes the step of pressurizing the heated stillage concentrate to prevent boiling. The method further includes recovering the oil from the treated stillage concentrate using a gravity separation process including centrifugation. The process disclosed by Winsness, et al. does not include treatment of un-concentrated stillage streams. While separation of stillage may be improved and oil can be recovered from the method of Winsness, et al., the process disclosed does not include recovery of a high solids-high protein fraction.

U.S. Pat. No. 6,106,673 to Walker discloses a process and system for the separation of a fermentation process byproduct into its constituent components and for the subsequent recovery of those constituent components. The process requires 1) mixing a starting mixture containing ethanol byproducts with a liquid (water) to form a diluted mixture, 2) heating of the diluted mixture containing the byproducts so as to separate the oil from a base component (fiber) of the byproduct to which the oil is bound at a temperature from about 140 degrees F. to about 250 degrees F., followed by 3) recovering oil, the base product (fiber), and possibly other substances such as molasses from the mixture. Although Walker recognizes that protein is contained in the recovered fibers, Walker teaches that the thermal treatment time and temperature should be limited so as to avoid protein breakdown, preferably no more than three minutes at 200 degrees F.

Woods, et al. in U.S. Pat. No. 8,609,874 assigned to Edeniq Inc. claims the recovery of oil from stillage including thin stillage and syrup and including the steps of heating and chemical treatment with alkali to raise the pH prior to recovering oil. Woods et al. do not claim the further recovery of a protein-enriched fraction after separation of the stillage.

Thus, there are recognized disadvantages and limitations of the prior art. Consistently, the deleterious impact of solubles, especially glycerol and minerals on the handling characteristics and stability of the dried feed product are not anticipated in the prior art. Due to the absence of hydrothermal treatment, the prior art is not able to produce a uniquely transformed hydrophobic protein agglomerate having a combination of high protein content (>45 weight % on a dry matter basis), low moisture adsorption characteristics, good shelf-life and stability and a desirable nutritional profile. Front-end fractionation methods are expensive to deploy at existing dry grind ethanol plants and the inefficiency of dry starch fractionation leads to lower starch yields and proportionally lower ethanol yields. In many back-end fractionation methods, oil is recovered with heat treatment but it is not recognized or anticipated that a high protein stream may be isolated. Other back end processes require costly chemicals or additives of limited nutritional value. Those back-end methods involving separation prior to distillation can result in ethanol yield loss due to the presence of residual ethanol in the wet proteinaceous solids and subsequent loss of the residual ethanol during protein drying. While some processes assert that they recover yeast cells, only a small portion of the yeast cells are recovered because the prior art processes do not induce agglomeration. Other methods disclose heat treatment as a precursor to separation for oil and fiber; however, they fail to disclose the further separation, isolation, and recovery of a valuable high-protein fraction.

Therefore, there is a need for an easily recovered protein product derived from a fermentation process that has high levels of grain and fermentation agent protein, reduced fat content, an improved amino acid profile and low levels of glycerol, minerals and other soluble solids.

### SUMMARY OF THE INVENTION

The present invention provides for a protein product recovered from a fermentation process including a protein content of 45.0% or more calculated by weight of dry matter, a glycerol content of 1.0% or less calculated by weight of dry matter, and a mineral nutrient content of 6.0% or less calculated by weight of dry matter.

The present invention also provides for a method for recovering a protein product by heating fermentation stillage to 200 degrees F.-350 degrees F., altering the physicochemical properties of the stillage to enable facile separation of the stillage, and separating a phase enriched in protein. The enriched protein phase may be further dewatered into a paste and dried.

The present invention further provides for a method for recovering a protein product by separating stillage into a first stream and a second stream, stripping protein from solids of the first stream, combining the solids of the first stream with an aqueous solution to create a third stream, separating the third stream into a fourth stream relatively high in protein and a fifth stream relatively low in protein, heating the second stream to 200 degrees F.-350 degrees F., altering the physicochemical properties of the stillage to enable facile separation of the stillage, and separating a phase enriched in protein from the second stream.

The present invention also provides for a method for recovering a protein product by separating stillage into a first stream and a second stream, stripping protein from the solids of the first stream, combining the solids of the first stream with an aqueous solution to create a third stream, separating the third stream into a fourth stream relatively high in protein and a fifth stream relatively low in protein, heating the fourth stream to 200 degrees F.-350 degrees F., altering the physicochemical properties of the stillage to enable facile separation of the stillage, and separating a phase enriched in protein from the fourth stream.

The present invention also provides for protein products, protein paste, high protein meal, and proteinaceous agglomerates recovered and formed in the above methods.

### DESCRIPTION OF THE DRAWINGS

Other advantages of the present invention are readily appreciated by reference to the following detailed description when considered in connection with the accompanying drawings wherein:

FIG. 1 is a flow diagram of a prior art ethanol fermentation process;

FIG. 2 is a flow diagram of the hydrothermal treatment process;

FIG. 3 is a flow diagram of a method to produce the product of the present invention with hydrothermal treatment as shown in FIG. 2 and further including separating, dewatering and drying the protein product;

FIG. 4 is a flow diagram of a method to produce the product of the present invention including a whole stillage shearing step prior to separation;

FIG. 5 is a flow diagram of a method to produce the product of the present invention including separating whole stillage into wet cake and thin stillage and then further separating the thin stillage, with aid of a counter-current wash process, into a second cut wet cake and a second cut thin stillage;

FIG. 6 is a flow diagram of a method to produce products of the present invention including separating stillage into a stream rich in source grain protein and an aqueous phase rich in fermentation agent and then dewatering the aqueous phase to recover the fermentation agent;

FIG. 7 is a flow diagram of a method to produce products of the present invention including separating stillage into a first stream and a second stream; grinding or shearing the first stream followed by mixing with an aqueous stream to produce a third stream, separating the third stream into a fourth stream containing recovered protein and a fifth stream;

FIG. 8 presents the particle size distribution of thin stillage, heat treated thin stillage and stickwater;

FIG. 9 is a series of photomicrographs indicating the presence of yeast in whole stillage and thin stillage and the preferential fractionation of yeast into the product of the present invention; and

FIG. 10 shows photographs of centrifuged tubes of thin stillage and heat treated thin stillage illustrating the facile separation of hydrothermally fractionated stillage under low-g centrifugation.

### DETAILED DESCRIPTION OF THE INVENTION

Most generally, the present invention provides for a protein product. The protein product's composition and physical properties makes it highly desirable as a feed product, and has good shelf life, good flowability, and high amino acid digestibility. The product compositions can include protein having elevated levels of essential amino acids, yeast biomass, lipid soluble components including tocopherols, and sterols, and low levels of glycerol and nutritional minerals. The present invention provides for proteinaceous agglomerates including denatured protein having exposed hydrophobic cores agglomerated with particles of similar hydrophobicity, and being easily separated and having improved dewatering characteristics. Preferably, the product compositions and proteinaceous agglomerates are recovered from a fermentation process. The present invention also relates to methods of recovering such protein products.

“Good shelf life” as used herein, refers to the product composition's ability to remain fit for use and the ability to not absorb moisture that allows microorganisms such as fungi and bacteria to grow. Good shelf life results from a decreased content of glycerol and nutrient minerals. Good shelf life of the product composition of the present invention results in reduced degradation and contamination compared to other protein products in the prior art.

“Good flowability” as used herein, refers to the ability of the product composition to flow. Factors in determining good flowability include particle size, size distribution, shape, surface area, and density. Flowability is reduced when moisture enters a product. Because of the low levels of glycerol in the product composition of the present invention, flowability is increased over prior art protein products.

“High amino acid digestibility” as used herein, refers to the ability of an animal to digest a high percentage of the amino acids found in a feed. The higher the amount of amino acids able to be digested, the more effective the feed and the more amino acids that the animal is able to receive. The product composition of the present invention is able to provide higher amino acid digestibility than prior art protein products.

“Proteinaceous agglomerates” as used herein, refers to clusters of proteins and similar particles held together by weak physical interactions. Proteins are hydrophobic, and therefore, they agglomerate with similar hydrophobic particles and other proteins. Hydrophilic particles can be excluded from the hydrophobic agglomerates. The proteins can also form micelles, i.e. spheres of the proteins, wherein hydrophobic ends of the proteins face one direction, and hydrophilic ends of the proteins face the opposite direction, depending on the solution they are in. For example, in a hydrophilic solution, the hydrophilic ends contact the solution, whereas the hydrophobic ends cluster together in the center of the micelle. Preferably, the proteinaceous agglomerates have protein with low levels of glycerol and nutrient minerals.

“Hydrophobic cores” as used herein, refers to the hydrophobic ends of the proteins clustered together in the center of the proteinaceous agglomerates, such that the center is hydrophobic.

“Easy separation” as used herein, refers to the ability of the proteinaceous agglomerates to separate readily from other compounds found in stillage. For example, the proteinaceous agglomerates can easily separate from the oil and stickwater fractions. This saves money and energy in the production of protein products.

“Improved dewatering characteristics” as used herein, refers to the ability of the proteinaceous agglomerates to have water and other liquid removed from them more readily than protein compositions of the prior art. This saves money and energy in the production of protein products.

“Stillage” as used herein, refers to a cloudy aqueous slurry produced during ethanol fermentation that includes solids that are not fermentable, solubles, oils, organic acids, salts, proteins, fermentation agent, and various other components. As described in the Background Art, in conventional dry-grind corn ethanol operations the effluent stillage from the bottom of the beer column is known as “whole stillage” which is then separated by centrifugation into “wet cake” and “thin stillage.”

“Hydrothermal fractionation” as used herein, refers to heating a substantially aqueous stillage stream to a temperature within a prescribed temperature range, and holding at temperature for a period of time within a prescribed residence time range, optionally cooling the stillage stream and then separating by gravity. A saturation pressure is established and maintained during the hydrothermal fractionation step.

“Physicochemical alteration” as used herein, refers to both physical and chemical changes that are imparted to the stillage by the hydrothermal fractionation step. Manifest physical changes include changes in the rate of phase separation owing to changes in relative phase densities and liquid phase viscosity changes in average size of particle agglomerates, changes in phase hydrophobicity and changes in color or appearance. Chemical changes include changes in the distribution of non-soluble protein, fat (oil) and carbohydrate (fiber) between the substantially liquid phase and the substantially solids phase. Other chemical changes include solublization and/or hydrolysis of components to increase the levels bio-available protein and ammonia in the soluble phase. These physical and chemical changes are mutually dependent and hence the term physicochemical is applied.

Stillage is a complex mixture of both hydrophobic and hydrophilic compounds. Hydrophobic compounds have no dipole moment and thus prefer other neutral molecules. Examples of hydrophobic compounds in stillage are lipids, lipid soluble compounds, and some proteins. Hydrophilic compounds have a pronounced dipole moment and thus prefer other charged molecules. Examples of hydrophilic compounds in stillage are water, glycerol, organic acids, nutrient minerals, and other ions. One advantage of the present invention is that hydrothermal treatment of stillage produces a physicochemical alteration that changes the nature of the soluble and insoluble components enabling a facile separation for improved recovery of co-products. The thermal energy imparted to the stillage denatures the proteins causing conformational changes exposing their hydrophobic core. Upon cooling, the proteins agglomerate with particles of similar hydrophobicity, i.e. hydrophobic compounds. These larger agglomerates are more easily separated and have improved dewatering characteristics. Furthermore, other components of the liquid phase, namely glycerol and nutrient minerals, are excluded from the agglomerates. These properties remain after drying to give the recovered product improved handling and storage characteristics. Yeast contains protein components both within and on the surface of the cell wall. The hydrothermal treatment exposes the hydrophobic core of these proteins. Increased hydrophobicity correlates with the propensity to flocculate through surface and transmembrane cell-to-cell interaction. Therefore, the hydrothermal treatment of the present invention enhances the recovery of yeast.

While heating of stillage has been performed as described in the prior art for recovery of corn oil and other by-products, it was not recognized that the hydrothermal treatment of stillage according to the present invention imparts physicochemical changes enabling facile separation into oil and an enriched protein stream and that the enriched protein stream can be dewatered and optionally dried to produce a superior animal feed component.

“Stickwater” as used herein, refers to a fraction of the hydrothermally treated stillage stream that is generally very low in suspended solids, typically less than 1 wt % or less than 50% of the suspended solids in conventional thin stillage, and is mainly water and solubles. “Stillage Protein Product” as used herein, refers to a proteinaceous fraction of the hydrothermally treated stillage stream that contains greater than 40 wt % of protein on a dry weight basis.

“Fermentation” as used herein, refers to a biological process, either anaerobic or aerobic, in which suspended or immobilized micro-organisms or cultured cells in a suitable media are used to produce alcohols, organic acids, other metabolites and/or new biomass.

“Fermentation agent” as used herein, refers to organisms that are used in the fermentation process to convert sugars to the targeted fermentation product. The organisms can be yeast, bacteria, algae, fungi, or some other organism, and combinations thereof. Preferably, a portion of the protein product and/or the proteinaceous agglomerates is from the fermentation agent.

“Nutritional minerals” as used herein refer to sulfur, phosphorus, potassium, magnesium, calcium, sodium, iron, manganese, copper, and zinc.

“Nutritional mineral content” as used herein, refers to the total dry weight of nutritional minerals limited to phosphorus, potassium, magnesium, calcium, sodium, iron, manganese, copper, and zinc, expressed as a percentage of total dry weight.

More specifically, the protein product preferably contains a protein level of 45% or more, and more preferably 48% or more, calculated by weight of dry matter. Protein is a critical nutrient for all species of animals. Protein deficiency results in loss of appetite, weight loss, and poor growth. While fiber and other non-protein product components are available across the world, protein production is concentrated in a few geographies. Efficient transportation of protein is critical. Fiber and other non-protein components have low bulk density making it expensive to transport. Therefore, high protein levels are desirable in an animal feed product.

The protein product preferably has low levels of glycerol, and more preferably, less than about 1.0% calculated by weight of dry matter. Glycerol is a simple polyol containing three hydroxyl groups. The hydroxyl groups are hydrophilic giving glycerol its hygroscopic nature, thus animal feed components that contain glycerol will attract moisture. This leads to poor shelf life and poor flowability.

The protein product can include a lysine content of greater than 1.5% calculated by weight of dry matter and/or a methionine content of greater than 0.5% calculated by weight of dry matter. Lysine and methionine are essential amino acids, meaning animals cannot synthesis these and must obtain them from their diets. Elevated levels of these amino acids are desirable in an animal feed.

The protein product can include about 10% or less fat by weight of dry matter. The protein composition can contain lipid soluble components including, but not limited to, lutein and zeaxanthin. Lutein and zeaxanthin can be present in contents each greater than 0.7 ppm calculated by weight of dry matter. Fat is a source of metabolizable energy and is desirable in an animal feed product. Fat in a protein source is desirable in lower limits such that the fat content does not diminish the protein content of the product and it does not reduce the inclusion rate of the product due to fat ration limitations. Lutein and zeaxanthin are naturally occurring carotenoids and are desirable in the diets of many animal species.

The protein product can include about 5.0% or greater yeast biomass by weight of dry matter. The yeast can be present in as much as 25% by weight of dry matter.

The protein product can include less than 6.0% nutritional mineral content calculated by weight of dry matter.

The protein product can include total tocopherols and sterols of at least 250 ppm by weight of dry matter.

The protein product can include at least 0.5% beta-glucan calculated by weight of dry matter.

The protein product can be dried when recovered and can contain a moisture content of less than 15% and more preferably less than 12% by weight of dry matter.

The protein product can also more specifically include a protein content of 45.0% or more calculated by weight of dry matter, a lutein content of greater than 0.7 ppm calculated by weight of dry matter, and a zeaxanthin content of greater than 0.7 ppm calculated by weight of dry matter.

Most preferably, the protein product and protein agglomerates of the present invention are recovered from a fermentation process. The following process details a method of producing the recovered product. However, it should be understood that the protein product and proteinaceous agglomerates can also be produced by other methods as well as synthesized.

Most generally, the present invention provides for a method for recovering a protein product, by heating fermentation stillage to 200 degrees F. to 350 degrees F., altering the physicochemical properties of the stillage to enable facile separation of the stillage, and separating a phase enriched in protein. Most preferably, the stillage is from a fermentation process. It should be understood though that the stillage in this method can also be other types besides fermentation stillage. The present invention also provides for the protein product recovered from this method.

The protein paste recovered from this method is described above. Most preferably, the protein paste includes at least 45 dry wt % protein, less than 1 dry wt % glycerol, amino acids in amounts chosen from the group consisting of at least 1.5 dry wt % lysine, 0.5 dry wt % methionine, and combinations thereof, less than 10 dry wt % fat and lipid soluble components, at least 5 dry wt % yeast biomass, less than 6 dry wt % nutritional minerals, 250 ppm dry wt tocopherols and sterols, as described above.

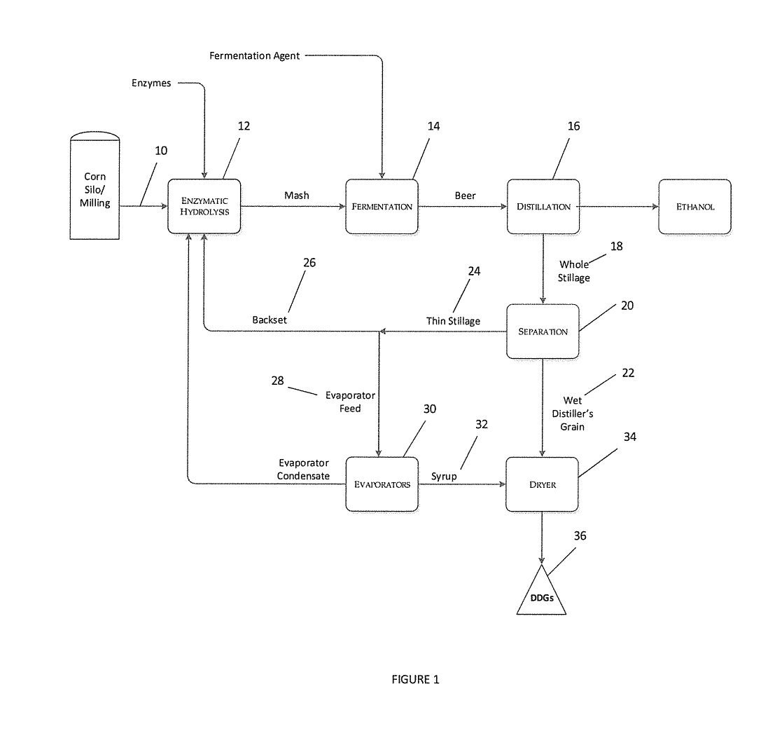

A depiction of a conventional dry-grind ethanol process is shown in FIG. 1. The hydrothermal treatment process can be utilized at various points in the process and on various stillage streams.

The stillage used in the method of the present invention can be whole stillage, thin stillage, clarified thin stillage, or thick stillage. Various types of stillage can be produced prior to the heating step of the hydrothermal treatment process. Thick stillage can be produced by methods such as, but not limited to, concentrating thin stillage, filtering whole stillage, centrifuging whole stillage under centrifuge operating conditions promoting transport of more solids into the centrate, adding solids to thin stillage, particle size reduction of stillage prior to filtration or centrifugation to increase the suspended solids in the feed to hydrothermal treatment, particle size reduction of grain or a grain slurry to increase the suspended solids in the feed to hydrothermal treatment, and combinations thereof. After thick stillage has been produced, some of the solids can additionally be removed. Clarified thin stillage can be produced by methods such as, but not limited to, filtration of thin stillage, centrifugation of thin stillage, and combinations thereof. Thin stillage can also be produced by separating stillage into a wet cake and thin stillage.

The particle size of the stillage can also be reduced by a mechanical size reduction device prior to the heating step, i.e. hydrothermal treatment process, of the method of the present invention. The size reduction device can be, but is not limited to, colloid mills, disc mills, pin mills, jet mills, rotor-stator mixers, high-pressure homogenizers, ultra-sonication, pluralities thereof, and combinations thereof.

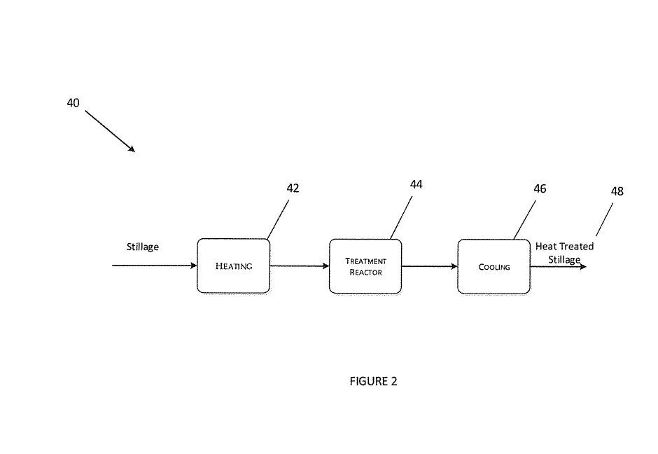

FIG. 2 shows the main steps of the hydrothermal treatment process. Stillage is heated by a heating mechanism (42), such as, but not limited to, a heat exchanger or steam injection, to a temperature of 200 degrees F. to 350 degrees F. and directed to a reactor (44). More preferably, the stillage is heated to 220 degrees F. to 300 degrees F. An Alfa Laval model TL10-PFG is an example of a plate and frame heat exchanger suitable to heat the entire stillage flow from a typical ethanol plant. The reactor pressure can be maintained at or above the saturation pressure of the stillage so as to avoid the loss of water vapor. The stillage is maintained at that temperature for 3 to 180 minutes. The reactor (44) can designate one or a plurality of reactors, operated batch or continuous and of any suitable design capable of maintaining the desired temperature, pressure and residence time. Agitation can be supplied as needed to prevent solids from settling in the reactor. Afterwards, preferably, the stillage is cooled (46) below its atmospheric boiling point, and preferably below 212 degrees F. Any suitable means can be used to cool the stillage including, but not limited to, a heat exchanger or flash cooler. It should be understood that in the case where the whole stillage has been previously heated to a temperature sufficient for hydrothermal treatment, for example during distillation at above atmospheric pressure, it may not be necessary to supply significant additional heat in the hydrothermal reactors. In this case the hydrothermal reactors simply provide sufficient residence time and maintain the target temperature (200 degrees F. to 350 degrees F.) through proper insulation and addition of relatively small amounts of maintenance heat.

The hydrothermal treatment step creates a new product, hydrothermally treated stillage (HTS) (48) that has unique physical and chemical properties significantly different from stillage. HTS has a less stable suspension of particles enabling facile separation and recovery of unique product fractions. HTS and these altered fractions accommodate a range of processing approaches. These fractions and products cannot be obtained in the prior art processes.

Due to this heating step, the stillage can readily separate even under quiescent settling conditions into a higher gravity solids fraction containing proteins, an aqueous stickwater fraction containing reduced suspended solids and a lighter oil phase. The physicochemical change imparted on the stillage by the heating step results in phase hydrophobicity, meaning that particles such as yeast cells and grain protein agglomerate into large particles or agglomerates. By the nature of the self-attraction, the agglomerates tend to exclude water and other hydrophilic components of the liquid phase such as glycerol and mineral nutrients.

These agglomerates have a higher settling velocity than non-agglomerated particles per the well-known Stokes equation for terminal settling velocity, Vₜ. In the Stokes equation below, rₚ and r_{f} are particle and fluid density respectively, g is gravitational acceleration, d is particle diameter and μ is fluid viscosity.

$${V}_{t} = \frac{{gd}^{2} ( {ρ}_{p} - {ρ}_{f} )}{18 μ} \qquad Stokes equation$$

Upon further examination of the Stokes equation, additional benefits of the present invention are apparent. Fluid density is reduced at elevated temperatures further enhancing the particle-fluid density difference (numerator). Owing to a combination of elevated temperature and lesser suspended solids, the viscosity of the fluid phase (denominator) is reduced, further contributing to increased sedimentation rates. Finally, the occurrence of gravitational acceleration (numerator) suggests the use of a high g-force device such as a centrifuge to further increase the sedimentation rate. Thus, while further mechanical partitioning processes can also be applied as described below, it is unexpected that merely by heating the stillage at this particular temperature range, the stillage can separate into the desired fractions.

In general, the amount of the separation due to the heat itself depends on the degree of solids removal prior to the hydrothermal fractionation step. If stillage with a low suspended solids level is used, the hydrothermal fractionation step readily induces separation. If whole stillage is used, the separation does not happen as readily as with thin stillage and whole stillage can therefore require a further mechanical partitioning or separation step as described below. Thus, in general, the heating step makes it easier to separate water from solids in the stillage regardless of the type of stillage used.

Generally, the altering of the physicochemical properties of the stillage includes changing the rate of phase separation owing to changes in relative phase densities and liquid phase viscosity, changes in average size of particle agglomerates, changing phase hydrophobicity and changes in color or appearance, changing the distribution of non-soluble protein, fat, and carbohydrate between the substantially liquid phase and the substantially solids phase, and solubilizing and hydrolyzing components to increase the levels bio-available protein and ammonia in the soluble phase. The altering step also includes altering the hydrophilic nature of the protein, exposing the hydrophobic core, and forming protein agglomerates that are easily separated and have improved dewatering characteristics.

Once the stillage has been hydrothermally treated to induce the physicochemical changes, multiple process schemes can be envisioned for separating the treated stillage into the product of the present invention. These schemes differ based on the type of equipment deployed, the residence time and the relative g-forces imparted by the specific equipment. Separation can be achieved with a method such as, but not limited to, gravity (quiescent decantation), screens, filtration, membranes, hydrocyclones, centrifugation, decanter centrifugation, dissolved air flotation, or any other suitable method, and pluralities thereof, and combinations thereof. Separation can also be performed with a single separation device such as a three-phase decanting centrifuge, a three-phase nozzle disc centrifuge, or a three-phase disk stack centrifuge. Any of these separation devices can be used in the separating steps described below. For example, the separation can be quiescent decantation for 10 to 180 minutes. Some examples of separation schemes are provided in the accompanying figures and are described below. Those skilled in the art will recognize that other schemes and equipment options can be utilized to arrive at the product of the present invention. Furthermore, it is not necessary to perform all separations and intermediate product compositions can be isolated if desired.

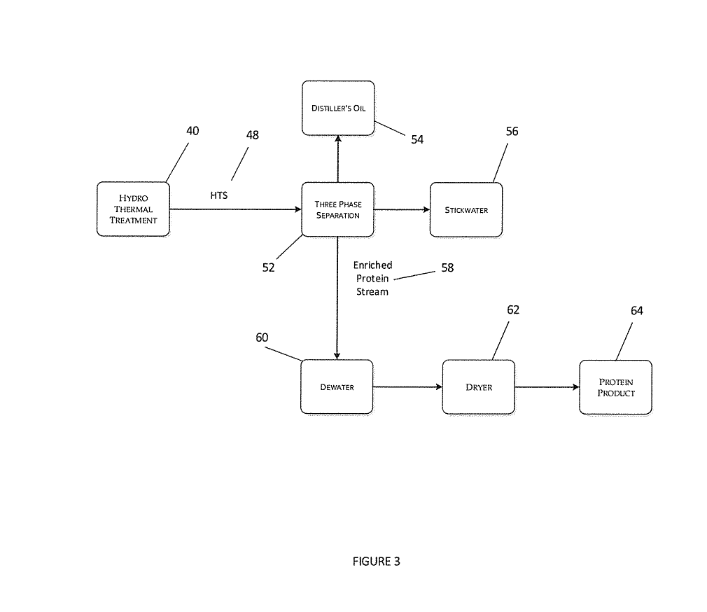

One method of recovering a protein product of the present invention is shown in FIG. 3. The HTS can be separated (52) by, for example, by a three-phase nozzle disc centrifuge into three streams; a distiller's oil stream (54), stickwater stream (56) and an enriched protein stream (58). A suitable device to perform such separation at a typical ethanol plant is a Valicor Model NDC-935 three-phase nozzle disc centrifuge to produce an enriched protein stream having about 18% total solids by weight. The enriched protein stream can be dewatered (60), for example in a decanting centrifuge to about 28% total solids by weight and optionally dried (62) into a protein product (64). Alternatively, the enriched protein can be simultaneously separated and dewatered from the hydrothermal treated stillage for example, in a two-phase decanter such as an Alfa Laval Decanting Centrifuge Model SG-700-2 with the recovery of distiller's corn oil from the HTS occurring either before or after the decanter. In general, dewatering can be performed by gravity settling, decanting centrifuge, disc stack centrifuge, nozzle disc centrifuge, filtering centrifuge, filters, membranes, hydrocyclones, partial evaporators, pluralities thereof (i.e. multiple numbers of the same type of device), and combinations thereof.

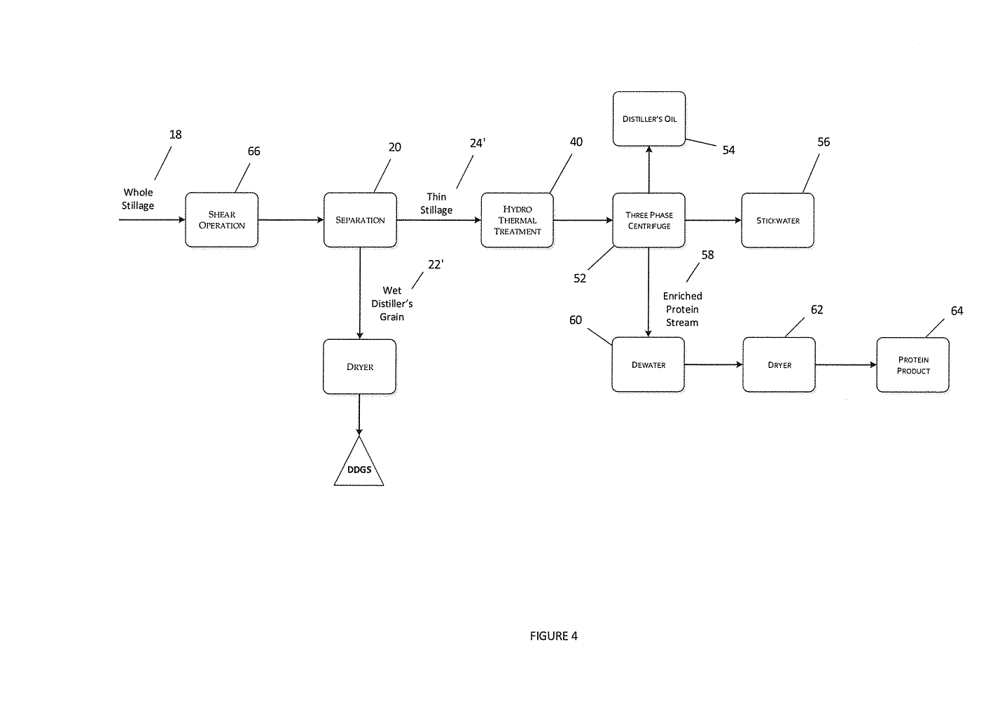

Various methods of processing either the whole stillage or thin stillage to further improve on the characteristics of the recovered product may be added. The whole stillage can be subjected to a shear device (66) prior to separation as shown in FIG. 4, or after separation. Shear devices include, but are not limited to, colloidal mills, grinders, refiners, shredders, macerators, grinding pumps, steam injectors, pluralities thereof, and combinations thereof. The shearing action helps strip additional protein from the stillage, increasing the amount available for recovery. The processed whole stillage can be separated into distiller's wet grains (22′) and thin stillage (24′), and the thin stillage can be hydrothermally treated (40), and separated into distiller's oil (54), stickwater (56), and an enriched protein stream (58). The enriched protein stream can be dewatered (60) and optionally dried (62) to produce a protein product (62). Therefore, prior to hydrothermal treatment, the stillage can be processed through a shear device.

Prior to the heating step, whole stillage can be separated by weight into a heavy phase and a light phase, and the light phase can be separated by size. The whole stillage can be separated by weight by a device such as a decanting centrifuge, disk stack centrifuge, hydrocyclones, dissolved air flotation, pluralities thereof, and combinations thereof. The light phase is separated by size by a device chosen from the group consisting of screens, pressure screens, filtering centrifuge, basket centrifuge, filters, membranes, pluralities thereof, and combinations thereof.

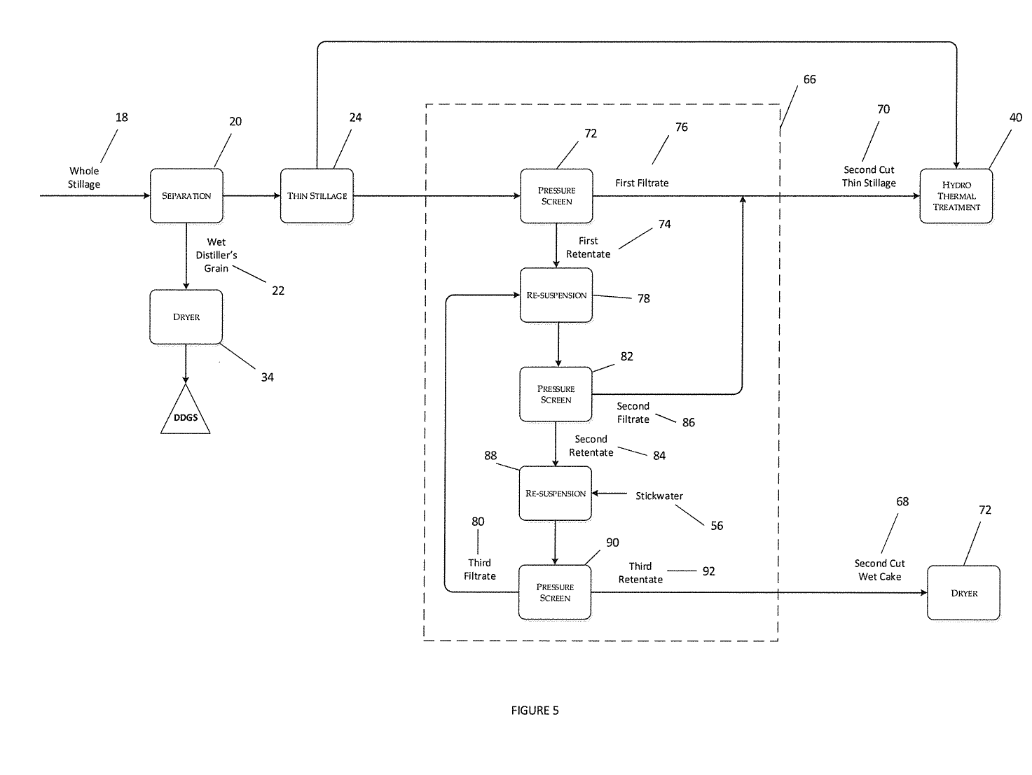

The present invention provides for a process as shown in FIG. 5. Stillage is separated into wet distiller's grains (22) and thin stillage (24). The thin stillage is separated (66) into a second cut wet cake (68) and a second cut thin stillage (70). A hydrocyclone, decanting centrifuge, filter, vibratory sieve, filtering centrifuge, paddle screen, static screen, pressure screen, nozzle disc centrifuge, disc stack centrifuge, decanting centrifuge, dissolved air flotation, membrane, or any other suitable devices can be used as the separation device. The two separations can use similar devices, such as two or more decanters in series or two or more filters in series. The two separations can use different devices. For example, the first separating device can be one or more decanting centrifuges and the second separating device can be one or more filters. In a typical ethanol plant, whole stillage can be processed through one or more Alfa Laval Model SG2-700 decanting centrifuges connected in parallel. The thin stillage can then be processed through one or more Dorr Oliver Pressure Fed 120 DSMR Screens. If more than one device is used to separate the whole stillage, the devices can be arranged in parallel or in series. If more than one device is used to separate the thin stillage, the devices can be arranged in parallel or in series. The second wet cake can be dried (72). The thin stillage and second cut thin stillage, either together or separately, can then be processed through the hydrothermal treatment (40) and subsequent separation.

Either or both of the separations can use a countercurrent wash system to increase the separation efficiency of the operation. FIG. 5 shows the use of countercurrent washing within a second cut separation system utilizing three pressure screens arranged in series; however any suitable devices may be used. The number of separation devices used can be two or more and often is a matter of economics. The greater the number of separation devices used, the more effective the separation; however, at some point, the incremental value of additional separation devices does not warrant the additional capital expense. Thin stillage is directed to a first pressure screen (72) where the stillage is separated into a first retentate (74) and a first filtrate (76). The retentate is re-suspended (78) with a third filtrate (80) and then directed to a second pressure screen (82) and separate into a second retentate ( 84) and second filtrate (86). The various pressure screens can have screens with the same size openings or different. The second retentate is re-suspended (88) with stickwater (56) or any other suitable wash water and directed to a third pressure screen (90) and separated into a third retentate (92) and a third filtrate (80). The third retentate can be recovered as second cut wet cake (68) and dried. Some or all of the first filtrate and some or all of the second filtrate can be combined to form the second cut thin stillage.

The process as described above has many desirable effects on the operation of the fermentation process and the attributes of the recovered products. The first separation can be performed in such a way that more suspended solids are directed to the thin stillage. The additional suspended solids will carry a greater amount of distiller's oil and protein for subsequent recovery. The process as described above facilitates other aspects of the present invention. For example, grinding or shearing can be introduced at various points in the process. Fine solids either inherent in the whole stillage or created by such grinding or shearing operations can be effectively removed by the secondary separation and thereby reduce solids loading in the downstream protein recovery centrifuge.

Therefore, prior to hydrothermal treatment, stillage can be separated into wet cake and thin stillage. The thin stillage can be separated into a second cut wet cake and a second cut thin stillage. In one embodiment, the separation devices are arranged in parallel of series. In another embodiment, the second cut wet cake is dried. The first cut thin stillage and second cut thin stillage, either together or separately, can then be processed through the hydrothermal treatment and subsequent separation as described above.

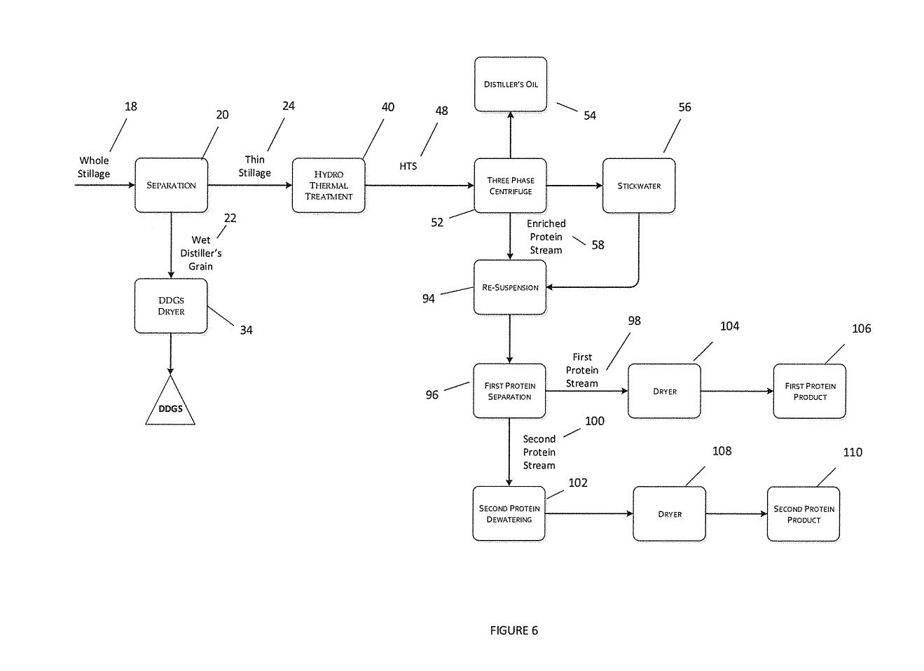

The present invention provides for a process as shown in FIG. 6, wherein whole stillage (18) is separated (20) into wet distiller's grain (22) and thin stillage (24) by, for example, a decanting centrifuge (decanter). The thin stillage undergoes hydrothermal treatment (40) by holding it at the target temperature for the prescribed amount of time. The HTS (48) is forwarded to a three-phase centrifuge (52) that separates the HTS into three streams; a distiller's oil stream (54), a stickwater stream (56) and an enriched protein stream (58) by, for example a disc stack centrifuge. At least some of the stickwater is used to re-suspend at least some of the enriched protein stream (94). The re-suspended slurry is subjected to a separation process (96) to remove a first protein stream (98) rich in grain protein and a second protein stream (100) rich in the fermentation agent. Any suitable device can be used such as a hydrocyclone, decanting centrifuge, or any other low g separation device. The second protein stream can be dewatered (102) using any suitable separation device such as a disc stack centrifuge or any other high g separation device. Distiller's oil can be removed simultaneously with the dewatering step with, for example with a three-phase nozzle disc centrifuge. The first protein stream can be dried (104) to a first protein product (106), preferably to less than 15 wt % moisture content. The second protein stream can be dried (108) to a second protein product (110), preferably to less than 15 wt % moisture content. The second protein stream produces a protein product enhanced in protein derived from the fermentation agent, and the first protein stream produces a protein product that has a lesser amount of protein content derived from the fermentation agent.

Alternatively, the HTS can be directed directly to the first protein separation without removing distiller's oil, such as in a two-phase decanting centrifuge. Distiller's oil can be removed simultaneously with the dewatering of the fermentation agent with, for example, a three-phase nozzle disc centrifuge.

Therefore, stillage can be hydrothermally treated and then separated into a first stream rich in grain protein and a second stream rich in a fermentation agent. The fermentation agent is recovered from the second stream. The first stream can be dewatered or dried. The fermentation agent can be dried. Oil can be removed prior to or simultaneous with the low g separation, or oil can be removed prior to or simultaneous with the high g separation.

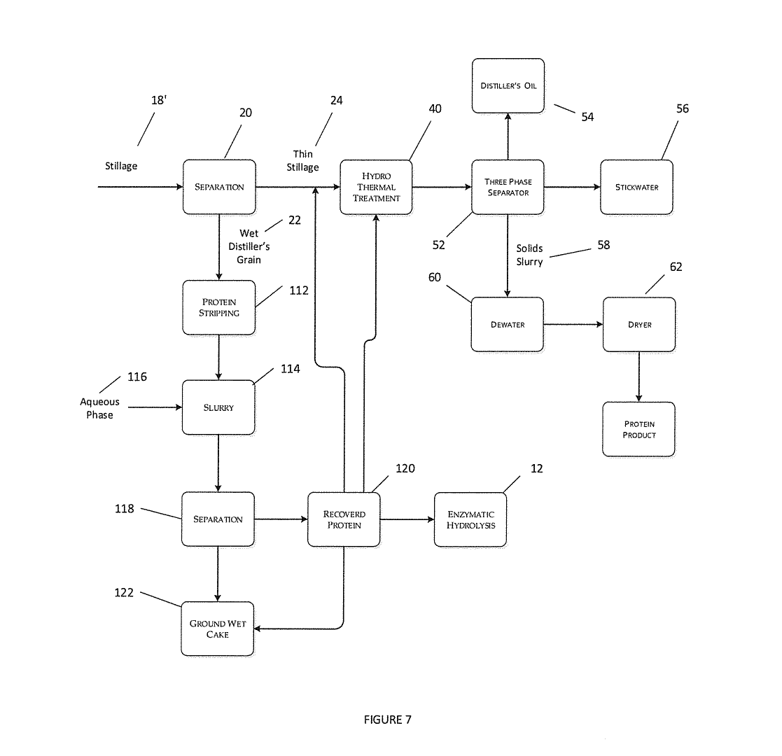

The present invention provides for a process as shown in FIG. 7, wherein stillage (18′) is separated (20) into a first stream containing wet distiller's grain (22) and thin stillage (24). Various processes may be added prior to the separation step. For example, whole stillage can be subjected to grinding, shearing or filtration to create stillage. The separation may be by any suitable means, such as but not limited to, a decanting centrifugation or filtration. The wet distiller's grain contains primarily large fibrous solids with attached protein. The wet distiller's grain is subjected to a shearing device or grinding device to strip protein from the fiber of the large solids and then slurried (114) with any suitable aqueous phase (116) to produce a third stream. The third stream is then separated (118) to produce a fourth stream containing recovered protein (120) and a fifth stream, ground wet cake (122). The protein in the fourth stream can be recovered at various points in the process. Some or the entire fourth stream can be returned to the enzymatic hydrolysis step (12) or other suitable step of the front end of the plant so that it can cycle through the fermentation and pass through to the stillage for subsequent recovery. Some or the entire fourth stream can be combined with some or the entire thin stillage stream (24). Some or the entire fourth stream can be hydrothermally treated (40), transforming the stream to allow for the recovery of a protein product of the present invention. While it is advantageous to recover the protein from the fourth stream, there can be times where this is impractical, for example, when this stream must be directed out of the fermentation process to maintain water balance. Therefore, some or the entire fourth stream can be added to the first stream, to the fifth stream or to other distiller's grain products. Some or the entire fourth stream can be evaporated.

Therefore, the present invention provides for a method wherein stillage is separated into a first stream including large solids and a second stream. Protein is stripped from the large solids of the first stream. The first stream is then re-slurried, i.e. solids of the first stream are combined with an aqueous solution, to produce a third stream. The third stream is separated into a fourth stream containing recovered protein (i.e. relatively high in protein) and a fifth stream (relatively low in protein). The second stream is heated to 200 degrees F.-350 degrees F., the physicochemical properties of the stillage are altered to enable facile separation of the stillage, and a phase enriched in protein is separated from the second stream.

In one embodiment, at least some of the fourth stream is recycled to the fermentation process. In one embodiment, at least some of the fourth stream is combined with at least some of the second stream. In one embodiment, at least some of the fourth stream is evaporated. In general, a step can be performed of combining some or all of the fourth stream with some or all of the second stream prior to the heating step, recycling some or all of fourth stream as make-up water, adding some or all of the fourth stream to the first stream, adding same or all of the fourth stream to the third stream, and combinations thereof. A phase enriched in protein from the fourth stream can be recovered, and the present invention provides for the protein product recovered by this method.

The present invention also provides for a method of recovering a protein product by separating stillage into a first stream and a second stream, stripping protein from the solids of the first stream, combining the solids of the first stream with an aqueous solution to create a third stream, separating the third stream into a fourth stream relatively high in protein and a fifth stream relatively low in protein, heating the fourth stream to 200 degrees F.-350 degrees F., altering the physicochemical properties of the stillage to enable facile separation of the stillage, and separating a phase enriched in protein from the fourth stream.

Additional amounts of soluble components such as glycerol, salts and minerals can be removed from the high protein fraction by methods known to those skilled in the art. For example, the high protein fraction from the three-phase high speed nozzle disc centrifuge in FIG. 2 can be “washed” by suspending and diluting in a water stream having significantly less solubles. The wash process can be performed with water containing less than half of the solubles found in the stickwater. The diluted stream can then be separated again (i.e. dewatered) by centrifuge, filtration, or micro-filtration membrane methods to remove dilution water containing soluble components. The wash step can be repeated as needed to achieve a targeted low level of solubles. Suitable water streams for wash purposes include steam condensate, municipal water or other treated water sources, e.g. process water treated by biological, chemical or adsorption and ion exchange methods. Other embodiments include performing the washing step in situ, i.e. within the confines of the centrifuge or filtration device. Such in situ washing devices are well known to those skilled in the art.

The recovered protein product can be dried by any suitable method known in the art. Preferably, the high protein meal/solids can be dried to a protein product having less than 15 wt % moisture. The recovered protein product can be further concentrated in evaporators. The present invention therefore provides for the protein product and high protein meal recovered from this method.

In summary, the unique hydrothermal treatment step of the present invention induces physicochemical changes that cause agglomeration of proteins and rejection of water and other components of the liquid phase. The present invention allows for the facile separation of stillage into valuable co-product fractions including, oil and stickwater and the product of the present invention, protein meal.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLES

Throughout the examples that follow, the product of the present invention may be referred to synonymously as “Stillage Protein Product”. The following analytical methods established by AOAC International, were used throughout multiple examples, shown in TABLE 1. Other methods are described within specific examples.

TABLE 1

| Analysis | AOAC Method # |
|---|---|
| Dry Weight or Total Solids | 934.01 (24 h, 105 deg C. method) |
| (w/w) | |
| Total Suspended Solids | 934.01 applied to the wet cake of a sample |
| | filtered through 2.2 μm filter media. |
| Amino Acid analysis | 994.12 |
| Neutral Fiber | 962.09E (neutral detergent fiber) |
| Crude Protein | 970.09 (Kjehldahl method) |
| Crude Fat/Oil | 920.39C Ether extraction method) |
| Minerals | AOAC 985.01 (ICP determination) |
| Free Glycerin | ASTM ® D6584 (GC Method) |

### Example 1

### Physicochemical Change Induced by Heat Treatment of Stillage

Procedures

This example demonstrates agglomeration of particles due to the hydrothermal treatment of thin stillage at conditions that promote protein denaturation, coagulation and agglomeration. For the present EXAMPLE 1, thin stillage obtained from a commercial ethanol plant was continuously pumped at a rate of 3 gallons per minute through a series of Plate and Frame Heat Exchangers (PHEs) into a 150 gallon stirred reactor maintained at saturation pressure and temperature. The PHEs heated the stillage to 250 degrees F. and the reactor had a mean residence time of 38 minutes. The hydrothermally treated stillage was cooled to approximately 150 degrees F. and fed at 3 gallons per minute to an Andritz model D2LC20C PC SA 3PH three-phase decanter centrifuge (“tricanter”). The decanter separated the heat-treated thin stillage into an oil (light phase), stickwater (medium phase), and protein-containing solids fraction (heavy phase).

Methods of Analysis

Samples of the initial thin stillage, heat-treated thin stillage and stickwater were sent to Fluid Imaging Technologies, Inc. for analysis with a FLOWCAM™ model VS4 image-based particle analyzer. For each sample, the sample container was vortexed to resuspend and mix the particles before analysis. Each sample was diluted in the following manner (with the dilution ratio accounted for in the software for determination of particle concentration of the original sample): 1 mL of original sample was brought to 40 mL in deionized water to make a first dilution and a second dilution was prepared by taking 1 mL of the first dilution to 40 mL again in deionized water. Samples of the second dilution were analyzed at 10× magnification in a 100 micro-liter flow cell.

Results and Discussion

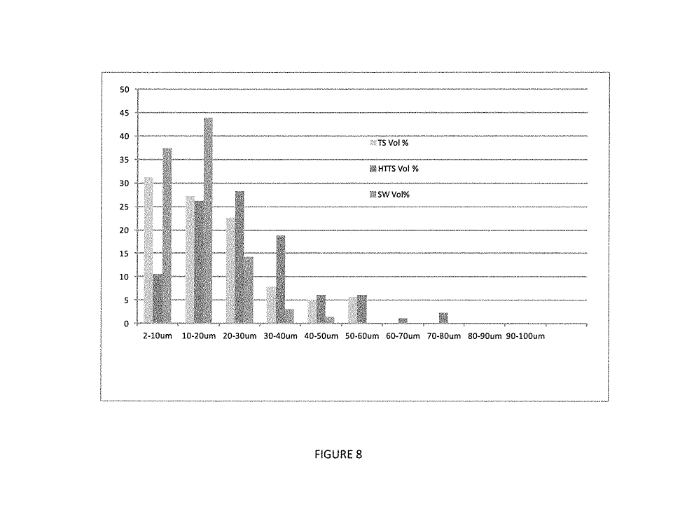

The particle size distribution for the Thin Stillage (TS), Heat Treated Thin Stillage (HTTS) and Stickwater (SW) samples as measured by FLOWCAM™ is shown in FIG. 8. Comparing TS and HTTS in FIG. 8, it is noted that the hydrothermal treatment of thin stillage results in a significant shift of the distribution to larger particle sizes. Subsequent centrifugation of HTTS removes the large particles creating a SW with a particle size distribution that is more similar to TS but having substantially reduced overall suspended solids. TABLE 2 below consolidates the data of FIG. 8 into simple mean/min/max particle area and diameter. The ABD Area is the number of pixels in a threshold (binary) greyscale two-dimensional image converted to a measure of area by use of a calibration factor. Diameter (ABD) is the diameter based on a circle with an area that is equal to the ABD Area. The minimum diameter of about 1 μm reflects the lower limit of resolution for the FlowCam imaging system. Of particular note in TABLE 2 is the shift in the maximum particle area from 916 μm2 for TS to 1786 μm2 for HTTS, providing quantitative evidence of the significant agglomeration caused by the hydrothermal treatment process.

TABLE 2
Summary statistics for Particle Area and Diameter

| | ABD AREA | | | DIAMETER | | |
|---|---|---|---|---|---|---|
| | (μm²) | | | (ABD) (μm) | | |
| Statistic | TS | HTTS | SW | TS | HTTS | SW |
| Mean | 11.8 | 24.8 | 13.9 | 3.27 | 4.30 | 3.53 |
| Min | 0.99 | 0.91 | 1.14 | 1.12 | 1.08 | 1.21 |
| Max | 917 | 1786 | 682 | 34.2 | 47.7 | 29.5 |
| StdDev | 27.0 | 67.8 | 24.1 | 2.08 | 3.61 | 2.27 |

### Example 2

### Determination of Protein Dispersability and Digestability

Procedures

For the present EXAMPLE 2, the protein dispersibility and digestibility of wet Stillage Protein Product and wet thin stillage solids are compared. Wet Stillage Protein Product was prepared as described in EXAMPLE 1. Wet thin stillage solids were prepared by collecting thin stillage from a commercial ethanol plant and feeding to a Valicor Model 1040 disc stack centrifuge. The disc stack supernatant was collected and discarded and the heavy phase was collected as thin stillage solids. Protein Dispersibility Index (PDI) was determined using the following method. The samples were suspended to the same total solids concentration in distilled water and mixed at a high speed for 10 minutes, then centrifuged. The supernatant was collected and analyzed for protein. The solubility of the protein is expressed as a percentage of the protein content of the supernatant divided by the protein content of the original sample. Protein digestibility measured as Pepsin Digestible Protein was determined by the following method. A dried sample was ground and mixed with a 35 ml solution of pepsin (1.5 mg/ml) in 0.1 M phosphate buffer (pH=2.0). The solution was incubated for 2 hours at 37 degrees C. The solution was centrifuged and the supernatant was collected and analyzed for protein.

Methods of Analysis

The AOAC analytical method for protein listed above was used in this example.

Results and Discussion

TABLE 3

| | Stillage | Thin |
|---|---|---|
| | Protein | Stillage |
| | Product | Solids |
| Protein Dispersibility Index (PDI) | 3.64% | 60.0% |
| (% as is) | | |
| Pepsin Digestible Protein (PDP) | 44.7% | 38.0% |
| (% of Dry Weight) | | |

As shown in TABLE 3, the PDI of the Stillage Protein Product is low which indicates the protein component of Stillage Recovered Product is minimally soluble in water, evidence of the hydrophobic surface character of the agglomerates. TABLE 3 also shows that denatured protein found in Stillage Protein Product is highly degradable by pepsin indicating a high level of digestibility. This example illustrates the utility of the process of the present invention to create a unique protein product having a hydrophobic character and low water solubility yet still retaining a high level of digestibility. These chemical characteristics are not evident in products obtained by prior art processes.

### Example 3

### Comparison of Proximate and Amino Acid Analysis of Stillage Protein Product to Other Feed Material

Procedures

For the present EXAMPLE 3, the crude protein, fiber, fat and ash concentrations of stillage protein product are compared to other corn based feed products. The stillage protein product was prepared by the following method. Whole stillage obtained from a commercial ethanol plant was filtered through a 300 micron screen. The 300 micron filtrate was then passed through a series of Plate and Frame Heat Exchangers (PHEs) into a stirred reactor. The PHEs heated the stillage to 240 degrees F. The reactor's pressure was maintained at the saturation pressure of the stillage. The reactor had a mean residence time of 40 minutes. The conditioned stillage was continuously withdrawn from the reactor and cooled to 185 degrees F. and pumped to a high speed disc stack centrifuge to remove the distiller's corn oil (DCO). The heavy phase and solids were collected and combined and pumped to a decanting centrifuge. The decanting centrifuge wet cake was collected, dried to 89% dry weight using a tray dryer at 200 degree F., and analyzed. DDGS and corn were obtained from a commercial corn ethanol plant. The corn gluten meal data was obtained from US Grain Council DDGS handbook, 3rd edition.

Methods of Analysis

The AOAC analytical methods listed above where used in this example.

Results and Discussion

TABLE 4 shows the protein, fat, fiber, and ash components of the various corn-based materials

TABLE 4

| | | Stillage | | Corn | |
|---|---|---|---|---|---|
| | | Protein | | Gluten | Whole |
| | | Product | DDGS | Meal | Corn |
| Dry Matter | % | 89.1 | 87.2 | 91.5 | 86.4 |
| Crude Protein | % dw | 50.6 | 26.5 | 66.3 | 9.7 |
| Crude Fat | % dw | 8.0 | 10.0 | 1.34 | 4.2 |
| Crude Fiber | % dw | 5.89 | 7.0 | 1.44 | 2.0 |
| Ash | % dw | 2.54 | 5.22 | 3.99 | 1.29 |

Stillage Protein Product has crude protein content much higher than whole corn or conventional DDGS and approaches corn gluten meal, a product of corn wet milling. Whole corn exhibits the lowest ash content by virtue of the fact that minerals become concentrated when other kernel fractions are removed or consumed during processing of the other materials; however, the Stillage Protein Product exhibits much lower ash content than DGGS or CGM due to the superior dewatering properties and enhanced removal of water soluble components including minerals. The samples were also analyzed for amino acid composition. The results are presented in TABLE 5 below. Stillage Protein Product contains high amounts of the essential amino acid lysine and essential sulfur containing amino acids methionine and cysteine, enhancing its value as an animal feed.

TABLE 5

| | | Stillage | | Corn | |
|---|---|---|---|---|---|
| | | Protein | | Gluten | |
| | | Product | DDG | Meal | Corn |
| Alanine | % | 3.7 | 2.1 | 5.5 | 0.7 |
| Arginine | % | 2.2 | 1.3 | 2.4 | 0.4 |
| Aspartic Acid | % | 3.6 | 1.9 | 4.2 | 0.6 |
| Cysteine | % | 0.9 | 0.5 | 1.1 | 0.2 |
| Glutamic acid | % | 7.6 | 4.4 | 13.5 | 1.8 |
| Glycine | % | 2.0 | 1.2 | 1.9 | 0.3 |
| Histidine | % | 1.4 | 0.8 | 1.4 | 0.3 |
| Isoleucine | % | 2.3 | 1.1 | 2.8 | 0.3 |
| Leucine | % | 6.4 | 3.5 | 10.7 | 1.5 |
| Lysine | % | 1.9 | 1.0 | 1.4 | 0.3 |
| Methionine | % | 1.1 | 0.6 | 1.4 | 0.2 |
| Phenylalanine | % | 2.8 | 1.3 | 4.1 | 0.5 |
| Proline | % | 4.0 | 2.1 | 5.6 | 0.8 |
| Serine | % | 2.2 | 1.4 | 2.9 | 0.4 |
| Threonine | % | 2.0 | 1.1 | 2.1 | 0.3 |
| Tryptophan | % | 0.4 | 0.2 | 0.2 | 0.1 |
| Tyrosine | % | 2.1 | 1.0 | 3.2 | 0.3 |
| Valine | % | 2.9 | 1.5 | 3.2 | 0.4 |

### Example 4

### Identification of Yeast in Stillage Protein Product

Procedures

For the present EXAMPLE 4, samples of whole stillage and thin stillage were obtained from a commercial ethanol plant. Stillage Protein Product and Stickwater were obtained per EXAMPLE 3. The samples where spread on glass slides and the water evaporate by heating. The samples were then subjected to the standard Gram Stain protocol.

Methods of Analysis

The samples were visualized and pictures taken using a Nikon 80i Digital Microscope at 400× magnification.

Results and Discussion

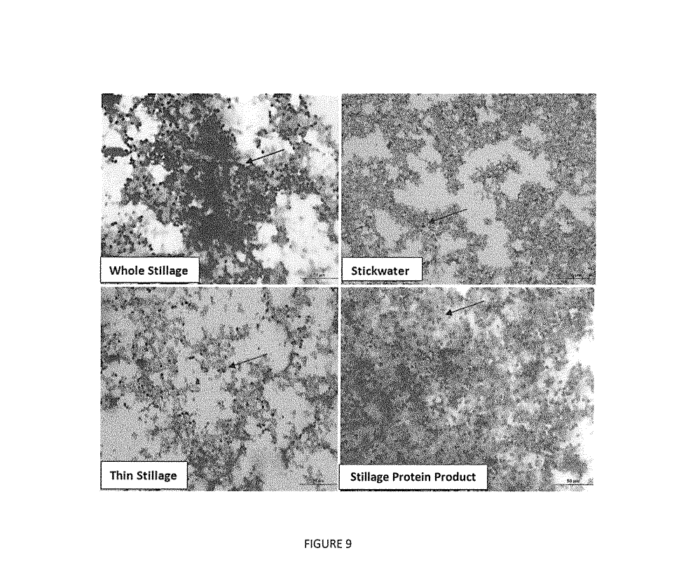

FIG. 9 shows representative photos of the samples after Gram staining. The dark oval circles indicated by the arrow are yeast cells. The results indicate that the yeast cells preferentially fractionate with the Stillage Protein Product and not stickwater.

### Example 5

### Determination of the Amount of Yeast Protein Present in Stillage Protein Product

Procedures

For the present EXAMPLE 5, whole stillage obtained from a commercial ethanol plant was filtered through a 300 micron screen. The 300 micron filtrate was then passed through a series of Plate and Frame Heat Exchangers (PHEs) into a stirred reactor. The PHEs heated the stillage to 240 degrees F. The reactor's pressure was maintained at the saturation pressure of the stillage. The reactor had a mean residence time of 40 minutes. The conditioned stillage was continuously withdrawn from the reactor and cooled to 185 degrees F. and pumped to a high-speed disc stack centrifuge to remove the distiller's corn oil (DCO). The heavy phase and solids were collected and combined and pumped to a decanting centrifuge. The decanting centrifuge wet cake was collected as a first cut Stillage Protein Product. The first cut Stillage Protein Product was dried to 89% dry weight using a tray dryer at 200 degree F., and analyzed. To dewater and recover a second cut Stillage Protein Product, the centrate from the decanting centrifuge was collected and centrifuged in 1-liter wide-mouth plastic bottles in a laboratory bottle centrifuge by ramping to full speed (3100 rpm, 2714 G-sec), holding for 5 min at full speed and then ramping down. DDGS were obtained from the same commercial ethanol plant. Thin stillage solids were prepared as described in EXAMPLE 4, and Stillage Protein Product TS was prepared as outlined in EXAMPLE 1. The amino acid profile was determined as outlined in EXAMPLE 3. A multiple linear regression as demonstrated by Han and Liu (J Agric Food Chem. 2010 Mar. 24; 58(6):3430-7.), was performed using the amino acid content of the various feeds to determine the amount of protein contributed to the product by corn and yeast.

Methods of Analysis

The AOAC analytical methods listed above where used in this example.

Results and Discussion

TABLE 6
Protein Content

| | | | | Second | |
|---|---|---|---|---|---|
| | First Cut | Stillage | | Cut | |
| | Stillage | Protein | | Stillage | Thin |
| | Protein | Product | | Protein | Stillage |
| | Product | TS | DDGS | Product | Solids |
| Corn | 81% | 48% | 85% | 53% | 48% |
| Yeast | 5% | 25% | 5% | 17% | 25% |
| Total | 86% | 73% | 89% | 70% | 73% |

As shown in TABLE 6 above, the amount of protein contributed by yeast to the Stillage Protein Product as determined by linear regression is about 5% in one embodiment of the process. In another embodiment of the process, in which the hydrothermal treatment is performed on conventional thin stillage, the yeast protein content is about 25% of the product. This example shows that embodiments of the present invention are capable of producing a range of products distinguished by the relative amounts of source protein present (grain and fermentation agent). The stillage protein product can thus be tailored to serve the needs of various feed markets and provide yet another option for by-product value enhancement by the grain ethanol plant. This flexibility has not been offered in the prior art.

### Example 6

### Nitrogen Corrected Total Metabolizeable Energy (TMEn) in Various Feed Materials

Procedures

For the present EXAMPLE 6, the TMEn of Stillage Protein Product was compared to that of DDGS and corn. Stillage Protein Product was prepared as described in EXAMPLE 3. A feed trial was conducted in which fasted cecectomized roosters were fed for two days. The treatment group was fed 30 grams per day of Stillage Protein Product and the control group was feed 30 grams per day of DDGS. The excreta were collected for analysis. The energy of the feed and excreta were determined by bomb calorimetry. The corn data was obtained from Poultry Science 87:2535-2548.

Methods of Analysis

The AOAC analytical methods listed above where used in this example.

Results and Discussion

TABLE 7 below indicates the TMEn of various protein containing feed materials.

TABLE 7

| | Stillage | | |
|---|---|---|---|
| | Protein | | |
| | Product | DDGS | Corn |
| TMEₙ (kCal/Kg dw) | 3500 | 2741 | 3411 |

Stillage Protein Product has a higher TMEn than DDGS or corn. Coupling this higher energy density with the superior protein content and amino acid profile demonstrated in Example 5, Stillage Protein Product is able to improve the overall value of by-products from a grain ethanol plant.

### Example 7

### Amino Acid Digestibility of Stillage Protein Produce and Corn Based Feeds

Procedures

For the present EXAMPLE 7, the amino acid digestibility of Stillage Protein Product was compared to the amino acid digestibility of DDGS and corn. The precision fed-cecectomized rooster assay (PFR) was conducted in which 30 grams/day of Stillage Protein Product or 30 grams/day of DDGS was fed to 8 fasted cecectomized roosters for two days. The amino acid profile of the Stillage Protein Product and DDGS and excreta was determined. The amino acid digestibility of corn and corn gluten meal were taken from published data (Poultry Science 91:3141-3147).

Methods of Analysis

The feed trials were conducted as referenced in Poultry Science 91:3141-3147. The AOAC analytical methods listed above where used in this example.

Results and Discussion

TABLE 8 below indicates the percent digestibility of each amino acid for various corn protein containing feed materials by poultry.

TABLE 8

| | | | |
|---|---|---|---|
| Stillage | | | Corn |
| Protein | | | Gluten |
| Product | DDGS | Corn | meal |

Non-Essential

| | | | | |
|---|---|---|---|---|
| Alanine | 86.8 | 82.7 | 91.7 | 95.8 |
| Aspartic Acid | 79.4 | 71.7 | 88.2 | 91.2 |
| Cysteine | 76.6 | 64.2 | 94.0 | 87.9 |
| Glutamic acid | 87.7 | 80.9 | 92.5 | 95.8 |
| Proline | 86.6 | 80.5 | 93.5 | 94.9 |
| Serine | 84.0 | 79.4 | 92.6 | 92.4 |
| Tyrosine | 88.5 | 82.8 | 86.3 | 92.4 |

Essential

| | | | | |
|---|---|---|---|---|
| Arginine | 89.6 | 84.7 | 91.5 | 93.1 |
| Histidine | 87.2 | 83.9 | 76.4 | 89.5 |
| Isoleucine | 86.6 | 81.8 | 88.2 | 93.4 |
| Leucine | 89.5 | 85.5 | 94.7 | 96.8 |
| Lysine | 80.5 | 72.5 | 74.5 | 84.2 |
| Methionine | 90.2 | 87.0 | 93.2 | 96.5 |
| Phenylalanine | 89.0 | 85.7 | 92.7 | 95.5 |
| Threonine | 80.9 | 74.1 | 90.8 | 90.8 |
| Valine | 84.4 | 77.8 | 87.4 | 92.6 |

% Digestibility

| | | | | |
|---|---|---|---|---|
| Average | 85.5 | 79.7 | 89.3 | 92.7 |
| Average Essential | 86.4 | 81.4 | 87.7 | 92.5 |
| Average Non- | 84.2 | 77.4 | 91.3 | 92.9 |
| Essential | | | | |

Stillage Protein Product has a higher amino acid digestibility than DDGS and a similar essential amino acid digestibility as corn. Again, the benefits of Stillage Protein Product produced by the method of the present invention are apparent. The hydrothermal treatment process enhances protein recovery and the physicochemical altered meal has improved nutritional attributes over that of the typical post fermentation DDG meal.

### Example 8

### Effect of Glycerol Addition on Moisture Gain by Stillage Protein Product

Procedures

Stillage Protein Product was obtained using in the process as described in EXAMPLE 1 and suspended in water. Glycerol was added in amounts as described in TABLE 9 below. The samples were then dried at 105° F. overnight. The samples were crushed and placed in aluminum weigh boats and weighed. A controlled environment of 90% humidity was created by filling the lower portion of a desiccator with saturated barium chloride solution. The samples were placed on a sample shelf suspended above the salt solution. The samples were incubated in the desiccator at room temperature for 5 days. The samples were then weighed and the percent weight gain was determined using astatic gravimetric method.

Methods of Analysis

The AOAC analytical methods listed above where used in this example.

Results and Discussion

TABLE 9 below indicates the moisture absorption at various glycerol additions. As the results indicate, increased levels of glycerol result in increased rates of moisture absorption as manifest by the sample weight gain. This is yet another example of the utility of the present invention. The product of the present invention has low glycerol levels and therefore a low rate of water absorption, long shelf life and good flowability.

TABLE 9

| Added | |
|---|---|
| Glycerol (by | |
| weight of dry | 5-day weight |
| matter) | gain (%) |
| 0.59 | 25.5 |
| 1.03 | 27.6 |
| 1.62 | 29.5 |
| 2.15 | 30.9 |
| 4.08 | 32.0 |
| 8.18 | 32.5 |

### Example 9

### Nutrient Mineral and Glycerol Composition

Procedures

Stillage Protein Product was obtained using process described in EXAMPLE 1. DDGS were obtained from a commercial dry grind ethanol plant.

Methods of Analysis

The AOAC analytical methods listed above where used in this example.

Results and Discussion

TABLE 10 below reports the nutrient mineral and glycerol content for Stillage Protein Product and DDGS. As the data indicates, the Stillage Protein Product contains less hygroscopic minerals and free glycerol than DDGS. This example illustrates the utility of the process to create a unique protein product. The product of the present invention has unique hydrophobic characteristics that enable the product to exclude hygroscopic molecules such as nutrient minerals and glycerol to produce a product that has superior material handling.

TABLE 10

| | Stillage | |
|---|---|---|
| | Protein | |
| Mineral | Product | DDGS |
| Sulfur (Total) (% of Dry Weight) | 0.80 | 0.78 |
| Phosphorus (Total) (% of Dry Weight) | 0.73 | 0.99 |
| Potassium (% of Dry Weight) | 0.67 | 1.30 |
| Magnesium (% of Dry Weight) | 0.18 | 0.33 |
| Calcium (% of Dry Weight) | 0.06 | 0.03 |
| Sodium (Total) (% of Dry Weight) | 0.18 | 0.35 |
| Iron (ppm of Dry Weight) | 228 | 78.2 |
| Manganese (ppm of Dry Weight) | 11 | 17.3 |
| Copper (ppm of Dry Weight) | 9.2 | 5.4 |
| Zinc (ppm of Dry Weight) | 39.2 | 58.7 |
| Total (% of Dry Weight) | 2.62 | 3.78 |
| Glycerol (free, i.e. not lipid | 0.01 | >3 |
| associated) (% (w/w) | | |

### Example 10

### Determination of the Equilibrium Moisture Content of Stillage Protein Product and Thin Stillage Solids

Procedures

For the present EXAMPLE 10, Stillage Protein Product was prepared as in EXAMPLE 1. Thin stillage solids were prepared as in EXAMPLE 5. The samples were then dried at 105° F. overnight. The samples were crushed and placed in aluminum weigh boats and weighed. The lower portion of a table top desiccator was filled with saturated barium chloride to maintain a 90% relative humidity environment. The samples were placed on a sample shelf suspended about the salt solution. The samples were incubated in the desiccator at room temperature for 2 days. The samples were weighed again. The water absorption characteristic was determined by measuring the percent weight gained.

Methods of Analysis

The AOAC analytical methods listed above where used in this example.

Results and Discussion

As shown in TABLE 11, the amount of moisture adsorbed by thin stillage solids is higher than the amount of moisture gained by Stillage Protein Product solids under the same conditions. This example illustrates the utility of the process to create a unique protein (feed) product. Due to the process the Stillage Protein Product has hydrophobic characteristics that enable a reduced rate of moisture adsorption and hence lower equilibrium moisture content in a humid environment. Reduced equilibrium moisture content creates a product with material handling characteristics superior to products produced by prior art.

TABLE 11

% Weight Gain

| Elapsed | Thin Stillage | Stillage Protein |
|---|---|---|
| Days | Solids | Product |
| 2 | 36.5% | 24.4% |
| 10 | 52.9% | 36.8% |

### Example 11

### Facile Separation of Stillage Induced by Present Invention

Procedures

Thin stillage was obtained from a commercial ethanol plant. The thin stillage was collected at approximately 175 degrees F. Treated stillage was prepared by heating thin stillage to 280 degrees F. in a stirred 1-gallon batch reactor, held for 40 minutes at temperature and cooled to approximately 175 degrees F. Samples of the heat-treated thin stillage and thin stillage were centrifuged at a relatively low g-force of 400×G for 30 seconds in 15 mL centrifuge tubes.

Results and Discussion

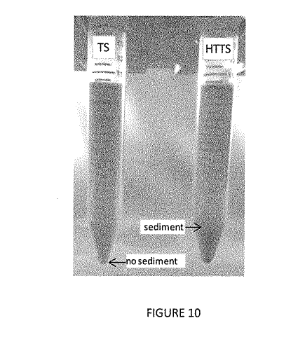

FIG. 10 shows that a clear line of sedimentation (settled solids) formed in the heat treated thin stillage sample whereas sediment was not observed in the untreated sample under the same short duration, low g-force conditions. Several factors induced by hydrothermal treatment contribute to the rapid sedimentation and dewatering of this novel proteinaceous material: (1) the increased particle size brought about by protein coagulation and inter-particle agglomeration, (2) the reduced water affinity of the particles due to exposed hydrophobic protein cores and (3) increased particle density by virtue of efficient unbinding of oil, oil having a specific gravity less than water.

This example illustrates the utility of the invention. A grain ethanol plant can recover a new, high value co-product that is significantly different than the current DDGS co-product. Again, due to the agglomeration of proteins and suspended solids as a result of the hydrothermal treatment process; proteins, fats, solubles, clarified aqueous phase (stickwater) and fibers are obtainable as separate products in amounts that would not otherwise be possible to obtain by prior art processes.

### Example 12

### Determination of the Dispersion Stability of Heat Treated Thin Stillage

Procedures

For EXAMPLE 12, samples of thin stillage and thin stillage heat treated as in EXAMPLE 1 were subjected to dispersion stability testing. The samples were well mixed and placed into a TURBISCAN™ (Formulation) measurement cell at room temperature and the transmission and back-scatter of near infrared (NIR) light was determined at intervals over a 5 hour time period. The TURBISCAN™ Stability Index (TSI) was calculated from the back-scatter NIR light data. TSI is expressed using the equation below:

$$T S I = {\sum }_{i} \frac{{\sum }_{h} \vert {scan}_{i} - {scan}_{i - 1} \vert}{H}$$

Where h is height of a scanned cell segment (approximately 40 um), “scan” is the intensity of backscattered NIR light, i is the scan number and H is the overall cell height. Thus TSI is a global measure of the cumulative back-scattered NIR light changes (scan-to-scan) for all segments over the total scan time. The greater the TSI value, the greater the light intensity changes and hence the worse the stability of the dispersion, or correspondingly, the greater the tendency of the particles to sediment.

Results and Discussion

TABLE 12

| Sample | TSI after 5 hours |
|---|---|
| Thin Stillage | 0.5 |
| Heat Treated Thin Stillage | 8.9 |

TABLE 12 above denotes that heat treated thin stillage has a higher TSI and is therefore a less stable dispersion than non-heat treated thin stillage. Analogous to Example 11 but in a quantitative manner, this example illustrates that heat treatment changes the physicochemical properties of the suspended particles producing agglomerates significantly different than that found in untreated stillage. This behavior of the molecules and particles from which a Stillage Protein Product is derived, has not been demonstrated in other prior art processes.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A protein product recovered from a fermentation process comprising:
a protein content of 45.0% or more calculated by weight of dry matter;
a glycerol content of 1.0% or less calculated by weight of dry matter; and
a mineral nutrient content of 6.0% or less calculated by weight of dry matter.

2. The protein product of claim 1, wherein the protein product has a protein content of 48% or more by weight of dry matter.

3. The protein product of claim 1, further comprising a lysine content of greater than 1.5% calculated by weight of dry matter, and a methionine content of greater than 1% calculated by weight of dry matter.

4. The protein product of claim 1, further comprising at least 5.0% yeast biomass calculated by weight of dry matter.

5. The protein product of claim 1, further comprising at least 0.5% beta-glucan calculated by weight of dry matter.

6. The protein product of claim 1, further comprising a total tocopherol and sterol content of at least 250 ppm by weight of dry matter.

7. The protein product of claim 1, further comprising 10% or less of combined fat and lipid soluble components by weight of dry matter.

8. The protein product of claim 7, wherein said lipid soluble components are chosen from the group consisting of lutein, zeaxanthin, and combinations thereof.

9. The protein product of claim 1, further comprising a moisture content of less than 15% by weight.

10. The protein product of claim 9, wherein said moisture content is less than 12% by weight.

11. A protein product recovered from a fermentation process comprising:
a protein content of 45.0% or more calculated by weight of dry matter;
a lutein content of greater than 0.7 ppm calculated by weight of dry matter; and
a zeaxanthin content of greater than 0.7 ppm calculated by weight of dry matter.

12. Proteinaceous agglomerates recovered from a fermentation process having improved separation and dewatering characteristics.

13. The proteinaceous agglomerates of claim 12, comprising protein that has low levels of glycerol and nutrient minerals.

14. The proteinaceous agglomerates of claim 13, wherein a portion of said protein is from the fermentation agent.

15. The proteinaceous agglomerates of claim 13, wherein said fermentation agent is chosen from the group consisting of yeast, bacteria, algae, fungi and combinations thereof.

16. A protein product comprising protein recovered from a fermentation process and having good shelf life, good flowability, and high amino acid digestibility.

17. A method for recovering a protein product, including the steps of:
heating fermentation stillage to 200 degrees F.-350 degrees F.;
altering the physicochemical properties of the stillage to enable facile separation of the stillage; and
separating a phase enriched in protein.

18. The method of claim 17, further including the step of dewatering the enriched protein phase to produce a protein paste.

19. The method of claim 18, wherein said dewatering step is performed by a method chosen from the group consisting of gravity settling, decanting centrifuge, disc stack centrifuge, nozzle disc centrifuge, filtering centrifuge, filters, membranes, hydrocyclones, partial evaporators, pluralities thereof, and combinations thereof.

20. The method of claim 18, wherein said dewatering step occurs simultaneously with said separating step.

21. The method of claim 18, wherein the protein paste is further defined as having on a dry matter basis at least 45% protein, less than 1% glycerol, amino acids in amounts chosen from the group consisting of at least 1.5% lysine, 0.5% methionine, and combinations thereof, at least 7% fat and lipid soluble components, at least 5% yeast biomass, less than 6% nutritional minerals, and 250 ppm tocopherols and sterols.

22. The method of claim 17, wherein said heating step is further defined as heating fermentation stillage to 220 degrees F.-300 degrees F.

23. The method of claim 17, wherein said heating step is performed for 3-180 minutes.

24. The method of claim 17, wherein heating is supplied only as needed to maintain the temperature of fermentation stillage which arrives at the hydrothermal treatment system at the target treatment temperature of 200 degrees F.-350 degrees F.

25. The method of claim 17, wherein said altering step is further defined as changing the rate of phase separation owing to changes in relative phase densities and liquid phase viscosity, changing the average size of particle agglomerates, changing phase hydrophobicity and changes in color or appearance, changing the distribution of non-soluble protein, fat, and carbohydrate between the substantially liquid phase and the substantially solids phase, solubilizing and hydrolyzing components to increase the levels of bio-available protein and ammonia in the soluble phase, and forming proteinaceous agglomerates.

26. The method of claim 17, further including, before said separating step, the step of cooling the heat-treated stillage to less than 212 degrees F.

27. The method of claim 17, wherein the stillage is chosen from the group consisting of whole stillage, thin stillage, clarified thin stillage, and thick stillage.

28. The method of claim 27, wherein the stillage is thick stillage produced by a method chosen from the group consisting of concentrating thin stillage, filtering whole stillage, centrifuging whole stillage under centrifuge operating conditions promoting transport of more solids into the centrate, adding solids to thin stillage, particle size reduction of stillage prior to filtration or centrifugation to increase the suspended solids in the feed to hydrothermal treatment, particle size reduction of grain or a grain slurry to increase the suspended solids in the feed to hydrothermal treatment, and combinations thereof.

29. The method of claim 28, further including the step of removing some of the solids from the thick stillage.

30. The method of claim 27, wherein the stillage is clarified thin stillage and is produced by a method chosen from the group consisting of filtration of thin stillage, centrifugation of thin stillage, pluralities thereof, and combinations thereof.

31. The method of claim 17, wherein the particle size of the stillage is reduced by a mechanical size reduction device prior to said heating step.

32. The method of claim 31, wherein the size reduction device is chosen from the group consisting of colloid mills, disc mills, pin mills, jet mills, rotor-stator mixers, high-pressure homogenizers, ultra-sonication, pluralities thereof, and combinations thereof.

33. The method of claim 17, wherein the stillage is subjected to a shearing device prior to said heating step.

34. The method of claim 33, wherein the shearing device is chosen from the group consisting of refiners, shredders, macerators, grinding pumps, steam injectors, pluralities thereof, and combinations thereof.

35. The method of claim 17, wherein said separating step is performed with a method chosen from the group consisting of gravity, screens, filtration, membranes, hydrocyclones, centrifugation, dissolved air flotation, pluralities thereof, and combinations thereof.

36. The method of claim 17, wherein said separating step is performed with a single separation device chosen from the group consisting of a three-phase decanting centrifuge, a three-phase nozzle disc centrifuge, and a three-phase disk stack centrifuge.

37. The method of claim 18, wherein the dewatering step is performed in a centrifugal decanter.

38. The method of claim 18, wherein additional solubles are removed from the enriched protein paste by a wash process performed with water containing less solubles than found in the stickwater.

39. The method of claim 38, wherein the wash process is chosen from the group consisting of suspending in low solubles water and dewatering, and washing with a device equipped with in situ wash capability.

40. The method of claim 17, further including the step of drying the enriched protein paste to a high protein meal having less than 15% moisture by weight.

41. The method of claim 17, further including prior to said heating step, the steps of:
separating whole stillage by weight into a heavy phase and light phase; and
separating the light phase by size.

42. The method of claim 41, wherein the whole stillage is separated by weight by a device chosen from the group consisting of decanting centrifuge, disk stack centrifuge, hydrocyclones, dissolved air flotation, pluralities thereof, and combinations thereof.

43. The method of claim 41, wherein the light phase is separated by size by a device chosen from the group consisting of screens, pressure screens, filtering centrifuge, basket centrifuge, filters, membranes, pluralities thereof, and combinations thereof.

44. The method of claim 17, wherein after said separating step, two protein product compositions are recovered by a process including the steps of:
separating the phase enriched in protein into two streams, the first stream having a greater concentration of protein derived from the fermentation agent than the second stream;
separating and dewatering the first stream to produce a protein product enhanced in protein derived from the fermentation agent; and
separating and dewatering the second stream to produce a protein product having a lesser amount of protein content derived from the fermentation agent than said first stream.

45. The method of claim 44, wherein said separating steps are performed with a method chosen from the group consisting of filtration, membranes, centrifugation, pluralities thereof, and combinations thereof.

46. The method of claim 44, wherein said separating steps are performed with a single separation device chosen from the group consisting of a three-phase decanting centrifuge, a three-phase nozzle disc centrifuge, and a three-phase disk stack centrifuge.

47. The method of claim 44, wherein the first protein product is dried to less than 15 wt % moisture content.

48. The method of claim 44, wherein the second protein product is dried to less than 15 wt % moisture content.

49. A method or recovering a protein product, including the steps of:
separating stillage into a first stream and a second stream;
stripping protein from solids of the first stream;
combining the solids of the first stream with an aqueous solution to create a third stream;
separating the third stream into a fourth stream relatively high in protein and a fifth stream relatively low in protein;
heating the second stream to 200 degrees F.-350 degrees F.;
altering the physicochemical properties of the stillage to enable facile separation of the stillage; and
separating a phase enriched in protein from the second stream.

50. The method of claim 49, further including a step chosen from the group consisting of combining some or all of the fourth stream with some or all of the second stream prior to said heating step, recycling some or all of fourth stream as make-up water, adding some or all of the fourth stream to the first stream, adding same or all of the fourth stream to the third stream, and combinations thereof.

51. The method of claim 50, further including recovering a phase enriched in protein from the fourth stream.

52. A method or recovering a protein product, including the steps of:
separating stillage into a first stream and a second stream;
stripping protein from the solids of the first stream;
combining the solids of the first stream with an aqueous solution to create a third stream;
separating the third stream into a fourth stream relatively high in protein and a fifth stream relatively low in protein;
heating the fourth stream to 200 degrees F.-350 degrees F.;
altering the physicochemical properties of the stillage to enable facile separation of the stillage; and
separating a phase enriched in protein from the fourth stream.

53. The protein product recovered from the method of claim 17.

54. The protein paste recovered by the method of claim 18.

55. The high protein meal recovered from the method of claim 40.

56. The protein product of claim 51.

57. The protein products of claim 44.

58. The high protein meal recovered from claim 47.

59. The high protein meal recovered from claim 48.

60. Proteinaceous agglomerates formed by the method of claim 25.
